(19) United States
(12) Patent Application Publication
(10) Pub. No.: US 2025/0152738 A1
(43) Pub. Date: May 15, 2025

(54) GPR17 PROMOTER-BASED TARGETING AND TRANSDUCTION OF GLIAL PROGENITOR CELLS

(71) Applicant: University of Rochester, Rochester, NY (US)

(72) Inventors: Steven A. Goldman, Webster, NY (US); Abdellatif Benraiss, Pittsford, NY (US); John Mariani, Rochester, NY (US)

(73) Assignee: University of Rochester, Rochester, NY (US)

(21) Appl. No.: 18/834,928

(22) PCT Filed: Feb. 1, 2023
(86) PCT No.: PCT/US2023/061751

§ 371 Date: Jul. 31, 2024

Related U.S. Application Data
(60) Provisional application No. 62/305,558, filed on Mar. 9, 2016.

(51) Int. Cl. A61K 48/00; C07K 14/47; C07K 14/705; C12N 15/86

(52) U.S. Cl. CPC A61K 48/005; C07K 14/47; C07K 14/705; C12N 15/86; C12N 2740/15043

(57) ABSTRACT

The present disclosure relates to a method of treating a disease or disorder in a subject in need thereof. This method involves providing a recombinant genetic construct comprising: (i) a G-protein coupled receptor (GPR17) promoter-inclusive regulatory element sequence having a 5′ and a 3′ end and (ii) a nucleic acid sequence encoding a molecule of interest, wherein said nucleic acid sequence is heterologous to the GPR17 promoter-inclusive regulatory element and wherein said nucleic acid sequence is positioned 3′ to the GPR17 promoter-inclusive regulatory element sequence; an expression vector comprising the recombinant genetic construct; a pharmaceutical composition comprising the recombinant genetic construct or the expression vector; or a preparation of cells comprising the recombinant genetic construct or the expression vector. This method further involves administering, to the subject in need, an effective amount of the recombinant genetic construct, the expression vector, the pharmaceutical composition, or the preparation of cells.

This application claims the priority benefit of U.S. Provisional Patent Application Ser. No. 63/305,558, filed Feb. 1, 2022, which is hereby incorporated by reference in its entirety.

This invention was made with government support under RO1 NS 110776 awarded by National Institutes of Health. The government has certain rights in the invention.

The Sequence Listing is being submitted electronically in XML format and is hereby incorporated by reference in its entirety. Said XML copy, created on Jan. 31, 2023, is named 147400-003871-SequenceListing.xml and is 24,282 bytes in size. No new matter is introduced

### FIELD

The present disclosure relates to GPR17 promoter-based targeting and transduction of glial progenitor cells.

### BACKGROUND

Oligodendrocytes are the sole source of myelin in the adult CNS, and their loss or dysfunction is at the heart of a wide variety of diseases of both children and adults. In children, the hereditary leukodystrophies accompany cerebral palsy as major sources of demyelination-associated neurological morbidity. In adults, demyelination contributes not only to diseases as diverse as multiple sclerosis and white matter stroke, but also to a broad variety of neurodegenerative and neuropsychiatric disorders (Lee et al., “Oligodendroglia Metabolically Support Axons and Contribute to Neurodegeneration,” Nature 487:443-448 (2012); Roy et al., “Progenitor Cells of the Adult White Matter,” In Myelin Biology and Disorders, R. Lazzarini, ed. (Amsterdam: Elsevier), pp. 259-287 (2004); Tkachev et al., “Oligodendrocyte Dysfunction in Schizophrenia and Bipolar Disorder,” Lancet 362:798-805 (2003)). As a result, the demyelinating diseases are especially attractive targets for cell-based therapeutic strategies.

Neurodegenerative disorders comprise a heterogeneous category, that include both multicentric and diffuse disorders such as Alzheimer's, and those in which the loss of a single phenotype predominates, such as Huntington's and Parkinson's diseases (Goldman, S. A., “Stem and Progenitor Cell-Based Therapy of the Central Nervous System: Hopes, Hype and Wishful Thinking,” Cell Stem Cell 18(2):174-188 (2016), which is hereby incorporated by reference in its entirety). The latter category, those neuronal disorders in which a single region or phenotype is differentially affected, have proven to be the most amenable to cell type-specific neuronal replacement in animal models (Lindvall, O., “Dopaminergic Neurons for Parkinson's Therapy,” Nature Biotechnology 30:56-58 (2012) and Lindvall & Bjorklund, “Cell Therapeutics in Parkinson's Disease,” Neurotherapeutics: The Journal of the American Society for Experimental Neuro Therapeutics 8:539-548 (2011)). These include classical Parkinson's disease, in which nigrostriatal neurons are lost before other neurons, and Huntington's disease, in which striatal atrophy becomes apparent long before the onset of more widespread cortical neuronal loss. Clinical trials of cell transplantation have already been performed for each of these prototypic neurodegenerative conditions (reviewed in (Barker et al., “Cell-Based Therapies for Parkinson Disease-Past Insights and Future Potential,” Nat. Rev. Neurol. 11:492-503 (2015); Barker et al., “The Long-Term Safety and Efficacy of Bilateral Transplantation of Human Fetal Striatal Tissue in Patients with Mild to Moderate Huntington's disease,” J. Neurol. Neurosurg. Psychiatry 84:657-665 (2013); Benraiss & Goldman, “Cellular Therapy and Induced Neuronal Replacement for Huntington's Disease,” Neurotherapeutics: The Journal of the American Society for Experimental Neuro Therapeutics 8:577-590 (2011); and Lindvall & Bjorklund, “Cell Therapeutics in Parkinson's Disease,” Neurotherapeutics: The Journal of the American Society for Experimental Neuro Therapeutics 8:539-548 (2011))). But these trials used fetal tissues dissected from the regions of interest, which thus included all cell types in the tissue, and not just the specific populations of nigrostriatal and striatal medium spiny neurons respectively lost in Parkinson's disease and Huntington's disease; in each of these cases, the target cell types typically comprised but a fraction of the cells delivered. Perhaps as a result, fetal tissue grafts into Parkinson's patients have yielded variable results, with both clear successes and failures, and a disturbingly high incidence of refractory dyskinesias, in which uncontrollable movements can negate the functional gains otherwise afforded by the grafted cells ((Barker et al., “Cell-Based Therapies for Parkinson Disease-Past Insights and Future Potential,” Nat. Rev. Neurol. 11:492-503 (2015)). Similarly, fetal striatal grafts into patients with Huntington Disease have yielded mixed results, with little evidence of significant or durable functional improvement (Cicchetti et al., “Neural Transplants in Patients with Huntington's Disease Undergo Disease-Like Neuronal Degeneration,” Proceedings of the National Academy of Sciences of the United States of America 106:12483-12488 (2009)).

Direct cellular reprogramming has allowed scientists to rapidly acquire cell types of interest for regenerative therapies and disease modeling (Lu & Yoo, “Mechanistic Insights Into MicroRNA-Induced Neuronal Reprogramming of Human Adult Fibroblasts,” Front. Neurosci. 12:522 (2018)). Several studies have demonstrated the direct conversion of human fibroblast cells to neuronal cells (see, e.g., Victor et al., “Generation of Human Striatal Neurons by microRNA-Dependent Direct Conversion of Fibroblasts,” Neuron 84(2): 311-323 (2014); U.S. Patent Application Publication No. 2002/0377885 to Yoo et al.; and Lu & Yoo, “Mechanistic Insights Into MicroRNA-Induced Neuronal Reprogramming of Human Adult Fibroblasts,” Front. Neurosci. 12: 522 (2018)). Empirically, however, obtaining mature human neurons from non-neuronal cells with transcription factors has been challenging (Caiazzo et al., “Direct Generation of Functional Dopaminergic Neurons from Mouse and Human Fibroblasts,” Nature 476(7359): 224-227 (2011)).

There remains a need for compositions that mediate glial progenitor cell-specific expression of therapeutic molecules, post-transcriptional modulators of gene expression, phenoconversion-promoting molecules, gene-editing molecules, and epigenetic editing molecules, as well as expression vectors, pharmaceutical compositions, preparations of cells, and methods of use thereof.

The present disclosure is directed to overcoming these and other deficiencies in the art.

### SUMMARY

A first aspect of the present disclosure relates to a method of treating a disease or disorder in a subject in need thereof. This method involves providing a recombinant genetic construct comprising: (i) a G-protein coupled receptor (GPR17) promoter-inclusive regulatory element sequence having a 5′ and a 3′ end and (ii) a nucleic acid sequence encoding a molecule of interest, wherein said nucleic acid sequence is heterologous to the GPR17 promoter-inclusive regulatory element and wherein said nucleic acid sequence is positioned 3′ to the GPR17 promoter-inclusive regulatory element sequence; an expression vector comprising the recombinant genetic construct; a pharmaceutical composition comprising the recombinant genetic construct or the expression vector; or a preparation of cells comprising the recombinant genetic construct or the expression vector. This method further involves administering, to the subject in need, an effective amount of the recombinant genetic construct, the expression vector, the pharmaceutical composition, or the preparation of cells. In some embodiments, the molecule of interest is selected from the group consisting of a therapeutic molecule, a post-transcriptional modulator of gene expression, a phenoconversion-promoting molecule, a gene-editing molecule, or an epigenetic editing molecule.

Another aspect of the present disclosure is directed to a recombinant genetic construct comprising: (i) a G-protein coupled receptor (GPR17) promoter-inclusive regulatory element sequence having a 5′ and a 3′ end and (ii) a nucleic acid sequence encoding a molecule of interest, wherein said nucleic acid sequence is heterologous to the GPR17 promoter-inclusive regulatory element and wherein said nucleic acid sequence is positioned 3′ to the GPR17 promoter-inclusive regulatory element sequence. In some embodiments, the molecule of interest is selected from the group consisting of a therapeutic molecule, a post-transcriptional modulator of gene expression, a phenoconversion-promoting molecule, a gene-editing molecule, or an epigenetic editing molecule.

Another aspect of the present disclosure is directed to an expression vector comprising a recombinant genetic construct according to the present disclosure.

A further aspect of the present disclosure is directed to a pharmaceutical composition comprising a recombinant genetic construct or an expression vector according to the present disclosure and a pharmaceutically acceptable carrier.

Another aspect of the present disclosure is directed to a preparation of cells comprising glial progenitor cells, where the cells of the preparation comprise a recombinant genetic construct or an expression vector according to the present disclosure.

Another aspect of the present disclosure relates to a method of delivering a nucleic acid construct encoding a protein of interest to glial progenitor cells. This method involves providing a recombinant genetic construct comprising: (i) a G-protein coupled receptor (GPR17) promoter-inclusive regulatory element sequence having a 5′ and a 3′ end and (ii) a nucleic acid sequence encoding the protein of interest, where the nucleic acid sequence encoding the protein of interest is heterologous to the GPR17 promoter-inclusive regulatory element and where the nucleic acid sequence encoding the protein of interest is positioned 3′ to the GPR17 promoter-inclusive regulatory element sequence. The method further involves transfecting or transducing the glial progenitor cells with the recombinant genetic construct.

Another aspect of the present disclosure relates to a method of delivering a nucleic acid construct encoding a protein of interest to astrocytes and glial progenitor cells. This method involves providing a recombinant genetic construct comprising: (i) a G-protein coupled receptor (GPR17) promoter-inclusive regulatory element sequence having a 5′ and a 3′ end; (ii) a glial fibrillary acidic protein (GFAP) promoter-inclusive regulatory element; and (ii) a nucleic acid sequence encoding the protein of interest, where the nucleic acid sequence encoding the protein of interest is heterologous to the GPR17 promoter-inclusive regulatory element as well as to the GFAP promoter-inclusive regulatory element and where the GFAP promoter-inclusive regulatory element is positioned within the recombinant genetic construct between the GPR17 promoter-inclusive regulatory element sequence and the nucleic acid sequence encoding the protein of interest. This method further involves transfecting or transducing a population of glial progenitor cells with the recombinant genetic construct, where (a) prior to differentiation of the transfected or transduced glial progenitor cells, the nucleic acid sequence encoding said protein of interest is expressed under control of the GPR17 promoter-inclusive regulatory element and (b) after differentiation of the transfected or transduced glial progenitor cells to astrocytes, said protein of interest is expressed under control of the GFAP promoter-inclusive regulatory element.

Bipotential oligodendrocyte-astrocyte and oligodendrocyte progenitor cells are a potential source of new myelinating oligodendrocytes in the human brain. As such they are potential targets for inducing, accelerating, and/or sustaining therapeutic remyelination. In addition, they are potential targets for exogenous transcription factor and/or CRISPR-mediated phenoconversion to neuronal lineages, of potential value in disorders as diverse as the vascular, neuroimmune, neurodegenerative, and neuropsychiatric diseases of neuronal loss. Yet all of these possible applications require the specific targeting of these cells, in humans, in vivo.

GPR17 is a G protein-coupled receptor that is expressed by glial progenitor cells during oligodendrocytic differentiation. Applicant has identified the GPR17 regulatory element that permits cell-specific expression of reporters and potentially therapeutic transgenes by human oligodendrocyte progenitor cells. Those cells that express GPR17 are destined for oligodendrocyte fate, and GPR17 is not expressed by any other brain cell phenotype. By placing the GPR17 promoter-inclusive regulatory element 5′ to genes of interest, and placing the resulting selection cassettes into viral vectors, Applicant has found that human oligodendrocyte-biased glial progenitor cells can be specifically targeted, both in vitro and in vivo, the latter in human glial chimeric mice.

This present disclosure provides a broad platform for the delivery of exogenous and synthetic genetic sequences including reporters, therapeutic transgenes, post-transcriptional modulators of gene expression such as miRNAs and shRNAs, phenoconversion-promoting sequences, and CRISPR/Cas-mediated genetic and epigenetic editing tools, among others, to glial and oligodendrocyte progenitor cells of the human brain, both in vitro/ex vivo and in vivo, and in both children and adult subjects.

### BRIEF DESCRIPTION OF THE DRAWINGS

FIGS. 1A-1D are schematic illustrations showing the design of lentiviral vectors comprising a recombinant genetic construct having a promoter-inclusive regulatory element and a nucleic acid sequence encoding enhanced green fluorescent protein (EGFP). The vectors further comprise a MIR123 target that allows silencing of the vector in neurons, as well as woodchuck hepatitis virus post-transcriptional regulatory element (WPRE). FIG. 1A shows a recombinant genetic construct comprising a glial fibrillary acidic protein (GFAP) promoter inclusive regulatory element sequence having a 5′ and a 3′ end and a nucleic acid sequence encoding EGFP, where the GFAP promoter inclusive regulatory element has the sequence of SEQ ID NO: 3. FIGS. 1B-1C show recombinant genetic constructs comprising: (i) a GPR17 promoter-inclusive regulatory element sequence having a 5′ and a 3′ end and (ii) a nucleic acid sequence encoding EGFP. FIG. 1B comprises a GPR17 promoter-inclusive regulatory element of SEQ ID NO: 1 (2.2 kb). FIG. 1C comprises a GPR17 promoter-inclusive regulatory element of SEQ ID NO: 2 (0.8 kb). FIG. 1D comprises a GPR17 promoter-inclusive regulatory element of SEQ ID NO: 1 (2.2 kb) and a GFAP promoter inclusive regulatory element of SEQ ID NO: 3. SA: Site acceptors; SD: Site donor; U5 and dU4: 5′ and 3′ Long terminal repeats of the lentivirus. MVM: Small intron of Minute virus mouse.

FIGS. 2A-2B demonstrate that lentiviral vector LV-GFAP-EGFP-M124T expression is restricted to Sox9 expressing astrocytes in the striatum. FIG. 2A is a schematic of the lentiviral vector LV-GFAP-EGFP-M124T. FIG. 2B are low magnification micrographs demonstrating that LV-GFAP-EGFP-M124T is expressed exclusively in Sox9-expressing astrocytes. Arrows indicate Sox9-expressing astrocytes. Scale bar: 25 μm.

FIGS. 3A-3B demonstrate that lentiviral vector LV-GPR17(2.2)-EGFP-M124T drives strong expression of EGFP reporter in vivo. FIG. 3A is a schematic of the lentiviral vector LV-GPR17(2.2)-EGFP-M124T. FIG. 3B are low magnification micrographs demonstrating the distribution of EGFP expressing cells in the striata of mice treated with lentiviral vector LV-GFAP-EGFP-M124T. Str: striatum; Ctx: cortex; CC: corpus callosum; scale bar: 500 25 μm.

FIGS. 4A-4B demonstrate that lentiviral vector LV-GPR17(2.2)-EGFP-M124T targets glial progenitor cells in vivo. FIG. 4A is a schematic of the lentiviral vector LV-GPR17(2.2)-EGFP-M124T. FIG. 4B are low magnification micrographs demonstrating that LV-GPR17(2.2)-EGFP-MT124 expression is restricted to glial progenitor cells (GPCs)/oligodendrocyte progenitor cells (OPCs) in adult mouse brain as show by colocalization of EGFP and PDGFRa double immuno-staining. Scale bar: 12 μm.

FIGS. 5A-5B demonstrate that lentiviral vector LV-GPR17(2.2)-EGFP-M124T targets glial progenitor cells in vivo. FIGS. 5A-5B are low magnification micrographs of cells demonstrating that GPR17 promoter-driven EGFP is expressed in OPCs co-expressing NG2 (FIG. 5A) and Olig2 (FIG. 5B), two specific markers that defines OPCs and oligodendroglia cells respectively. Scale bar: 20 μm.

FIG. 6 demonstrates that LV-GPR17(2.2)-EGFP-M124T targets glial progenitor cells in vivo. EGFP⁺/Olig2⁺ double immuno-stained cells (arrows) showed typical morphology of OPCs and young oligodendrocytes cells. Left panel should EGFP, Olig2, and DAPI fluorescence; right panel shows Olig3 and DAPI florescence. Scale bar: 50 μm.

FIGS. 7A-7B demonstrate that LV-GPR17(2.2)-EGFP-MT124 is not expressed in astrocytes or neurons in the adult mouse brain. FIGS. 7A-7B are low magnification micrographs showing EGFP-expressing cells in LV-GPR17(2.2)-EGFP-MT124-treated animal did not express the astrocytes marker ALDH1L1 (FIG. 7A) or the neuronal marker NeuN (FIG. 7B). Arrows indicate GFP⁺ cells. Scale bar: 20 μm.

FIGS. 8A-8B demonstrate that LV-GPR17(0.8)-EGFP-MT124 is equally as efficient at reporting human GPCs/OPCs as LV-GPR17(2.2)-EGFP-MT124. FIG. 8A is a schematic of the lentiviral vector LV-GPR17(0.8)-EGFP-MT124. FIG. 8B is a low magnification micrograph of striatum of LV-GPR17(0.8)-EGFP-MT124T-treated mice. Str: striatum; Ctx: cortex; CC: corpus callosum; Scale bar: 500 μm.

FIG. 9 are micrographs demonstrating that striatally-injected LV-GPR17(0.8)-EGFP-M124T expression is restricted to glial progenitor cells in vivo. 800 bp of the GPR17 promoter drives EGFP expression in Olig2⁺ oligodendroglial cells. Arrows indicate EGFP⁺/Olig2⁺ double positive cells. Scale bar: 50 μm.

FIG. 10 are micrographs demonstrating that LV-GPR17(0.8)-EGFP-M124T is not expressed in astrocytes of the adult mouse striatum. Arrows indicate lack of Sox9 expression by GFP⁺ cells. Scale bar: 50 μm.

FIGS. 11A-11B demonstrate that striatally-injected LV-GPR17(2.2)-GFAP-EGFP-M124T is expressed by both glial progenitor cells and astrocytes. FIG. 11A is a schematic of the lentiviral vector LV-GPR17(2.2)-GFAP-EGFP-M124T. FIG. 11B is a low magnification micrograph of striatum of a brain section of mice injected with LV-GPR17 (2.2)-GFAP-EGFP-M124T. Str: striatum; Ctx: cortex; CC: corpus callosum; Scale bar: 500 μm.

FIG. 12 are micrographs demonstrating that lentiviral vector LV-GPR17(2.2)-GFAP-EGFP targets mixed cell population of cells in the striatum. LV-GPR17-GFAP-EGFP-MT124 is expressed Sox9-immunopositive astrocytes (arrowheads) in the adult mouse striatum. However, EGFP expression is also depicted in non-astrocytic cell (Sox9 immuno-negative; Arrows). Scale bar: 50 μm.

FIGS. 13A-13B are micrographs demonstrating that LV-GPR17-GFAP-EGFP targets both GPCs and astrocytes in the striatum. LV-GPR17-GFAP-EGFP-MT124 injected striata showed EGFP expression that only partially colocalize with NG2-expressing glial progenitor cells (Arrow); arrowhead indicates NG2 immuno-negative cells (FIG. 13A). EGFP-expressing cells comprise both Sox9-immunopositive astrocytes (arrows) and Olig2-immunopositive cells of oligo-dendroglial lineage (arrowheads) (FIG. 13B). Scale bar: 50 μm.

FIGS. 14A-14C are graphs demonstrating the cell type specificity of promoter-based lentiviral targeting vectors. FIG. 14A is a bar graphs showing EGFP expressing cell distribution in strata injected with LV-GPR17(2.2)-EGFP or LV-GPR17(0.8)-EGFP. FIG. 14B is a bar graphs showing that LV-GFAP-EGFP-M124T drives expression predominantly in Sox9-expressing astrocytes. FIG. 14C is a bar graphs showing that dual promoter lentivirus LV-GPR17-GFAP-EGFP drives expression in both Astrocytes (Sox9*) and oligodendroglia lineage cells (Olig2⁺).

FIG. 15 is a schematic of pTANK-GFAP-EGFP-MIR124 (9042 bp).

FIG. 16 is a schematic of pTANK-GPR17(2.2)-EGFP-MIR124T (10,528 bp).

FIG. 17 is a schematic of pTANK-GPR17)0.8)-EGFP-MIR124T (9129 bp bp).

FIG. 18 is a schematic of pTANK-GPR17(2.2)-GFAP-EGFP-MIR124T (11,456 bp).

### DETAILED DESCRIPTION

### Definitions

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “and/or” as used herein means that the listed items are present, or used, individually or in combination. In effect, this term means that “at least one of” or “one or more” of the listed items is used or present.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

The term “complementary” when used in connection with nucleic acid, refers to the pairing of bases, A with T or U, and G with C. The term “complementary” refers to nucleic acid sequences that are completely complementary, that is, form A to T or U pairs and G to C pairs across the entire reference sequence, as well as molecules that are partially (e.g., at least 80%, 81%, 82%, 83%, 84%, 85%, 86%, 87%, 88%, 89%, 90%, 91%, 92%, 93%, 94%, 95%, 96%, 97%, 98%, or 99%) complementary.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### Methods of Treating a Disease or Disorder

A first aspect of the present disclosure relates to a method of treating a disease or disorder in a subject in need thereof. This method involves providing a recombinant genetic construct comprising: (i) a G-protein coupled receptor (GPR17) promoter-inclusive regulatory element sequence having a 5′ and a 3′ end and (ii) a nucleic acid sequence encoding a molecule of interest, wherein said nucleic acid sequence is heterologous to the GPR17 promoter-inclusive regulatory element and wherein said nucleic acid sequence is positioned 3′ to the GPR17 promoter-inclusive regulatory element sequence; an expression vector comprising the recombinant genetic construct; a pharmaceutical composition comprising the recombinant genetic construct or the expression vector; or a preparation of cells comprising the recombinant genetic construct or the expression vector. This method further involves administering, to the subject in need, an effective amount of the recombinant genetic construct, the expression vector, the pharmaceutical composition, or the preparation of cells.

In some embodiments, the molecule of interest is selected from the group consisting of a therapeutic molecule, a post-transcriptional modulator of gene expression, a phenoconversion-promoting molecule, a gene-editing molecule, or an epigenetic editing molecule.

Suitable subjects in accordance with the methods disclosed herein include, without limitation mammals, such as humans, non-human primates, rodents (e.g., mice or rats), rabbits, guinea pigs, cats, dogs, cows, birds, horses, sheep, pigs, and experimental animal models. In some embodiments, the subject is a human.

Suitable recombinant genetic constructs, expression vectors, pharmaceutical compositions, and preparations of cells for use in the methods according to the present disclosure are described in detail infra.

The recombinant genetic constructs according to the present disclosure, expression vectors according to the present disclosure, pharmaceutical compositions according to the present disclosure, and preparation of cells according to the present disclosure may be administered by parenteral, topical, oral or intranasal means to a subject in need thereof. In some embodiments of the methods according to the present disclosure, such recombinant genetic constructs, expression vectors, and pharmaceutical compositions are administered as a sustained release composition or device. In some embodiments, the recombinant genetic constructs, expression vectors, or pharmaceutical compositions disclosed herein are injected directly into a particular tissue, for example by intracranial injection.

The recombinant genetic construct, expression vector, pharmaceutical composition, or preparation of cells may be introduced into one or more sites of the subject's brain, brain stem, spinal cord, or a combination thereof. In some embodiments, the preparation of cells is transplanted bilaterally into multiple sites of the subject. Suitable methods of introducing recombinant genetic constructs, expression vectors, pharmaceutical compositions, and preparations of cells into one or more sites of the brain are well known to those of skill in the art.

In some embodiments, a recombinant genetic construct, an expression vector, a pharmaceutical composition, or a preparation of cells of the present disclosure is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In some embodiments the recombinant genetic constructs, expression vectors, or pharmaceutical compositions disclosed herein are administered by intravenous or subcutaneous injection or infusion.

In some embodiments, the recombinant genetic construct, expression vector, pharmaceutical composition, or preparation of cells is administered intraventricularly, intracallosally, or intraparenchymally.

In some embodiments, the recombinant genetic construct, expression vector, pharmaceutical composition, or preparation of cells is administered intraventricularly. In accordance with such embodiments, the recombinant genetic construct, expression vector, pharmaceutical composition, or preparation of cells is placed in a ventricle, e.g., a cerebral ventricle. For example, grafting cells in a cerebral ventricle may be accomplished by injection of the donor cells or by growing the cells in a substrate such as 30% collagen to form a plug of solid tissue which may then be implanted into the ventricle to prevent dislocation of the graft cells. For subdural grafting, the cells may be injected around the surface of the brain after making a slit in the dura.

In some embodiments, the recombinant genetic construct, expression vector, pharmaceutical composition, or preparation of cells is administered intracallosally as described in U.S. Patent Application Publication No. 20030223972 to Goldman, which is hereby incorporated by reference in its entirety. The recombinant genetic construct, expression vector, pharmaceutical composition, or preparation of cells can also be delivered directly to the forebrain subcortex, specifically into the anterior and posterior anlagen of the corpus callosum. The recombinant genetic construct, expression vector, pharmaceutical composition, or preparation of cells can also be delivered to the cerebellar peduncle white matter to gain access to the major cerebellar and brainstem tracts. The recombinant genetic construct, expression vector, pharmaceutical composition, or preparation of cells can also be delivered to the spinal cord.

In some embodiments, the recombinant genetic construct, expression vector, pharmaceutical composition, or preparation of cells is administered intraparenchymally. Intraparenchymal administration is achieved by injection or deposition of tissue within the brain so as to be apposed to the brain parenchyma at the time of transplantation. The two main procedures for intraparenchymal transplantation are: 1) injecting the recombinant genetic construct, expression vector, pharmaceutical composition, or donor cells within the host brain parenchyma or 2) preparing a cavity by surgical means to expose the host brain parenchyma and then depositing the recombinant genetic construct, expression vector, pharmaceutical composition, or donor cells into the cavity (Bjorklund and Stenevi (eds), Neural Grafting in the Mammalian CNS, Ch. 3, Elsevier, Amsterdam (1985), which is hereby incorporated by reference in its entirety). Both methods provide parenchymal apposition between the recombinant genetic construct, expression vector, pharmaceutical composition, or donor cells and host brain tissue at the time of grafting, and both facilitate anatomical integration between the recombinant genetic construct, expression vector, pharmaceutical composition, or donor cells and host brain tissue. This is of importance if it is required that the recombinant genetic construct, expression vector, pharmaceutical composition, or donor cells become an integral part of the host brain and survive for the life of the host.

In some embodiments, the subject has a disease or disorder selected from the group consisting of a vascular disorder, a neuroimmune disorder, a neurodegenerative disorder, and a neuropsychiatric disease of neuronal loss.

A neurodegenerative disease or neurodegenerative disorder is a chronic progressive neuropathy characterized by selective and generally symmetrical loss of neurons in motor, sensory, or cognitive systems. Exemplary neurodegenerative diseases and neurodegenerative disorders include, without limitation, Huntington's disease, degenerative dementia, Alzheimer's disease, frontotemporal dementia, Parkinson's disease, multisystem atrophy, amyotrophic lateral sclerosis, and conditions mediated by a deficiency in myelin. In some embodiments, the subject has a neurodegenerative disorder selected from the group consisting of Huntington's disease, frontotemporal dementia, Parkinson's disease, multisystem atrophy, and amyotrophic lateral sclerosis.

Huntington's disease is an autosomal dominant neurodegenerative disease characterized by a relentlessly progressive movement disorder with devastating psychiatric and cognitive deterioration. Huntington's disease is associated with a consistent and severe atrophy of the neostriatum which is related to a marked loss of the GABAergic medium-sized spiny projection neurons, the major output neurons of the striatum. Huntington's disease is characterized by abnormally long CAG repeat expansions in the first exon of the Huntingtin gene (“HTT”). The encoded polyglutamine expansions of mutant huntingtin protein disrupt its normal functions and protein-protein interactions, ultimately yielding widespread neuropathology, most rapidly evident in the neostriatum.

Alzheimer's disease (“AD”) is a progressive, degenerative brain disease that slowly erodes memory and thinking skills, and eventually even the ability to carry out simple tasks. It is the most common cause of dementia.

Frontotemporal dementia is a group of related conditions resulting from the progressive degeneration of the temporal and frontal lobes of the brain. These areas of the brain play a significant role in decision-making, behavioral control, emotion, and language.

Amyotrophic lateral sclerosis (ALS, commonly called “Lou Gehrig's disease”) is the most common motor neuron disease in adults. Motor neuron diseases are neurodegenerative diseases that cause selective loss of the nerve cells that directly connect the brain to muscles.

In some embodiments, the subject has a neuropsychiatric disorder. A neuropsychiatric disease or neuropsychiatric disorder is a chronic progressive neuropathy characterized by selective and generally symmetrical loss of neurons in motor, sensory, or cognitive systems. Exemplary neuropsychiatric diseases or neuropsychiatric disorders include, without limitation, schizophrenia, autism spectrum disorder, and bipolar disorder.

Schizophrenia is a serious mental illness that affects how a person thinks, feels, and behaves. The symptoms of schizophrenia generally fall into the following three categories: 1) psychotic symptoms including altered perceptions, 2) negative symptoms including loss of motivation, disinterest and lack of enjoyment, and 3) cognitive symptoms including problems in attention, concentration, and memory.

Autism spectrum disorder is a neurodevelopment disorder that causes a wide range of impairments in social communication and restricted and repetitive behaviors.

Bipolar disorder is a serious mental illness characterized by extreme mood swings. They can include extreme excitement episodes or extreme depressive feelings. Three types of bipolar disorder include: 1) Bipolar I Disorder, defined by manic episodes, 2) Bipolar II Disorder, that is defined by depressive episodes, and 3) Cyclothymic Disorder, defined by periods of hypomanic and depressive symptoms.

In some embodiments, the neurodegenerative disease is a myelin disease or a condition mediated by a deficiency in myelin. In some embodiments, the subject has a myelin disease. The myelin disease may be a leukodystrophy or a white matter disease. In some embodiments, the condition mediated by a deficiency in myelin may be selected from the group consisting of pediatric leukodystrophies, the lysosomal storage diseases, congenital dysmyelination, cerebral palsy, inflammatory demyelination, post-infectious and post-vaccinial leukoencephalitis, radiation- or chemotherapy induced demyelination, and vascular demyelination.

Leukodystrophy refers to a group of rare, primarily inherited neurological disorders known as the leukodystrophies that result from the abnormal production, processing, or development of myelin and other components of central nervous system (CNS) white matter, such as cells called oligodendrocytes and astrocytes. All leukodystrophies are the result of genetic defects (mutations).

In some embodiments, the condition mediated by a deficiency in myelin requires myelination. In other embodiments, the condition mediated by a deficiency in myelin requires remyelination. In some embodiments, the condition requiring remyelination is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, spinal cord injury, radiation- or chemotherapy induced demyelination, post-infectious and post-vaccinial leukoencephalitis, periventricular leukomalacia, and cerebral palsy.

In therapeutic applications (i.e., in applications involving a subject who has been diagnosed with a vascular, neuroimmune, neurodegenerative, or neuropsychiatric disease or disorder) recombinant genetic construct, expression vector, pharmaceutical composition, or preparation of cells of the present disclosure are administered to such a patient in an amount sufficient to cure, treat, or at least partially arrest the symptoms of the disease (as adduced by biochemical, histologic and/or behavioral assessment), including its complications and intermediate pathological phenotypes in development of the disease. In some embodiments, the administration of the recombinant genetic construct, expression vector, pharmaceutical composition, or preparation of cells of the present disclosure reduces or eliminates symptoms of the disease or disorder.

Effective doses of the provided recombinant genetic construct, expression vector, pharmaceutical composition, or preparation of cells of the present disclosure, for the treatment of the above-described diseases or disorders may vary depending upon many different factors, including means of administration, target site, physiological state of the patient, other medications administered. Treatment dosages are typically titrated to optimize their safety and efficacy. On any given day that a dosage is given, the dosage of the recombinant genetic construct, expression vector, or pharmaceutical composition of the present disclosure may range from about 0.0001 to about 100 mg/kg, and more usually from about 0.01 to about 5 mg/kg, of the patient's body weight. For example, dosages can be 1 mg/kg body weight or 10 mg/kg body weight or within the range of 1-10 mg/kg body weight. Exemplary dosages thus include: from about 0.1 to about 10 mg/kg body weight, from about 0.1 to about 5 mg/kg body weight, from about 0.1 to about 2 mg/kg body weight, from about 0.1 to about 1 mg/kg body weight, for instance about 0.15 mg/kg body weight, about 0.2 mg/kg body weight, about 0.5 mg/kg body weight, about 1 mg/kg body weight, about 1.5 mg/kg body weight, about 2 mg/kg body weight, about 5 mg/kg body weight, or about 10 mg/kg body weight.

A physician or veterinarian having ordinary skill in the art may readily determine and prescribe the effective amount of the recombinant genetic construct, expression vector, pharmaceutical composition, or preparation of cells required. For example, the physician or veterinarian could start doses of recombinant genetic construct, expression vector, or preparation of cells in the pharmaceutical composition at levels lower than that required in order to achieve the desired therapeutic effect and gradually increase the dosage until the desired effect is achieved. In general, a suitable daily dose will be that amount which is the lowest dose effective to produce a therapeutic effect. Such an effective dose will generally depend upon the factors described above. Administration may, e.g., be intravenous, intramuscular, intraperitoneal, or subcutaneous, and for instance administered proximal to the site of the target. If desired, the effective daily dose may be administered as two, three, four, five, six or more sub-doses administered separately at appropriate intervals throughout the day, optionally, in unit dosage forms. While it is possible the recombinant genetic construct, expression vector, pharmaceutical composition, or preparation of cells of the present disclosure to be administered alone, it is preferable to administer the recombinant genetic construct, expression vector, pharmaceutical composition, or preparation of cells of the present disclosure as a pharmaceutical composition as described above.

For therapeutic purposes, the recombinant genetic construct, expression vector, pharmaceutical composition, or preparation of cells of the present disclosure may be administered on multiple occasions. Intervals between single dosages (e.g., a bolus or infusion) can be weekly, monthly, or yearly. Alternatively, the therapeutic molecules of the present disclosure can be administered as a sustained release formulation, in which case less frequent administration is required. Dosage and frequency vary depending on the half-life of the recombinant genetic construct, expression vector, pharmaceutical composition, or preparation of cells of the present disclosure in the patient.

In another embodiment, a pharmaceutical composition comprising a recombinant genetic construct or expression vector as described herein, is administered to a subject to facilitate in vivo expression of the therapeutic molecule, the post-transcriptional modulator of gene expression, the phenoconversion-promoting molecule, the gene-editing molecule, or the epigenetic editing molecule for the treatment of a disease or disorder described herein (e.g., Huntington's disease, degenerative dementia, Alzheimer's disease, frontotemporal dementia, Parkinson's disease, multisystem atrophy, amyotrophic lateral sclerosis, or a condition mediated by a deficiency in myelin). Expression vector constructs suitable for use in this embodiment of the disclosure are described infra.

The recombinant genetic construct, expression vector, pharmaceutical composition, or preparation of cells of the present disclosure can result in the generation of the therapeutic molecule, the post-transcriptional modulator of gene expression, the phenoconversion-promoting molecule, the gene-editing molecule, or the epigenetic editing molecule in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The recombinant genetic construct, expression vector, pharmaceutical composition, or preparation of cells of the present disclosure can result in generation of the therapeutic molecule, the post-transcriptional modulator of gene expression, the phenoconversion-promoting molecule, the gene-editing molecule, or the epigenetic editing molecule in the subject within at least about 1 day, 2 days, 3 days, 4 days, 5 days, 6 days, 7 days, 8 days, 9 days, or 10 days of administration of the composition to the subject. The recombinant genetic construct, expression vector, pharmaceutical composition, or preparation of cells of the present disclosure can result in generation of the therapeutic molecule, the post-transcriptional modulator of gene expression, the phenoconversion-promoting molecule, the gene-editing molecule, or the epigenetic editing molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the recombinant genetic construct, expression vector, or pharmaceutical composition of the present disclosure to the subject.

The recombinant genetic construct, expression vector, pharmaceutical composition, or preparation of cells of the present disclosure, when administered to the subject in need thereof, can result in the persistent generation of the therapeutic molecule, the post-transcriptional modulator of gene expression, the phenoconversion-promoting molecule, the gene-editing molecule, or the epigenetic editing molecule in the subject. The recombinant genetic construct, expression vector, pharmaceutical composition, or preparation of cells can result in the generation of the therapeutic molecule, the post-transcriptional modulator of gene expression, the phenoconversion-promoting molecule, the gene-editing molecule, or the epigenetic editing molecule in the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

In some embodiments, administering the recombinant genetic construct, expression vector, pharmaceutical composition, or preparation of cells of the present disclosure is effective to treat the disease or disorder in the subject. In accordance with such embodiments, “treating” the disease or disorder encompasses: (1) preventing, delaying, or reducing the incidence and/or likelihood of the appearance of at least one clinical or sub-clinical symptom of the state, disorder, or disease developing in a subject that may be afflicted with or predisposed to the state, disorder, or disease, but does not yet experience or display clinical or subclinical symptoms of the state, disorder, or disease; or (2) inhibiting the state, disorder, or disease, i.e., arresting, reducing or delaying the development of the disease or a relapse thereof or at least one clinical or sub-clinical symptom thereof, or (3) relieving the disease, i.e., causing regression of the state, disorder, or disease or at least one of its clinical or sub-clinical symptoms. The benefit to a subject to be treated is either statistically significant or at least perceptible to the subject or to the physician.

In some embodiments, a preparation of cells of the present disclosure is administered to the subject. Suitable preparations and methods of administering such preparations are described in more detail supra. In some embodiments, the glial progenitor cells of the administered preparation are astrocyte biased glial progenitor cells, oligodendrocyte-biased glial progenitor cells, unbiased glial progenitor cells, or a combination thereof. The glial progenitor cells of the administered preparation may express one or more markers of the glial cell lineage. For example, in one embodiment, the glial progenitor cells of the administered preparation may express A2B5⁺. In another embodiment, the glial progenitor cells of the administered preparation are positive for a PDGFαR marker. The PDGFαR marker is optionally a PDGFαR ectodomain, such as CD140a. PDGFαR and CD140a are markers of an oligodendrocyte-biased glial progenitor cells. In another embodiment, the glial progenitor cells of the administered preparation are CD44⁺. CD44 is a marker of an astrocyte-biased glial progenitor cell. In another embodiment, the glial progenitor cells of the administered preparation are positive for a CD9 marker. The CD9 marker is optionally a CD9 ectodomain. In one embodiment, the glial progenitor cells of the administered preparation are A2B5⁺, CD140a⁺, and/or CD44⁺. The aforementioned glial progenitor cell surface markers can be used to identify, separate, and/or enrich the preparation for glial progenitor cells prior to administration.

The administered glial progenitor cell preparation is optionally negative for a PSA-NCAM marker and/or other neuronal lineage markers, and/or negative for one or more inflammatory cell markers, e.g., negative for a CD11 marker, negative for a CD32 marker, and/or negative for a CD36 marker (which are markers for microglia). Optionally, the preparation of glial progenitor cells are negative for any combination or subset of these additional markers. Thus, for example, the preparation of glial progenitor cells is negative for any one, two, three, or four of these additional markers.

In some embodiments, the preparation is administered to one or more sites of the brain, the brain stem, the spinal cord, or a combination thereof.

In some embodiments, the preparation is administered intraventricularly, intracallosally, or intraparenchymally.

In accordance with the method that involves administering the preparation of cells according to the present disclosure, the selected preparation of cells may comprise at least about 80% glial progenitor cells, including, for example, about 80%, 85%, 90%, 95%, 96%, 97%, 98%, 99%, 100% glial cells. The selected preparation of cells can be relatively devoid (e.g., containing less than 20, 15, 10, 9, 8, 7, 6, 5, 4, 3, 2, or 1%) of other cells types such as neurons or cells of neuronal lineage). Optionally, example cell populations are substantially pure populations of glial progenitor cells.

Delivery of the preparation of cells to the subject can include either a single step or a multiple step injection directly into the nervous system. Multiple injections sites can be performed to optimize treatment. Injection is optionally directed into areas of the central nervous system such as white matter tracts like the corpus callosum (e.g., into the anterior and posterior anlagen), dorsal columns, cerebellar peduncles, cerebral peduncles. Such injections can be made unilaterally or bilaterally using precise localization methods such as stereotaxic surgery, optionally with accompanying imaging methods (e.g., high resolution MRI imaging). One of skill in the art recognizes that brain regions vary across species; however, one of skill in the art also recognizes comparable brain regions across mammalian species.

The preparations of cells may be optionally injected as dissociated cells but can also be provided by local placement of non-dissociated cells. In either case, the preparation of cells may optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated cellular transplant can be a streaming injection made across the entry path, the exit path, or both the entry and exit paths of the injection device (e.g., a cannula, a needle, or a tube). Automation can be used to provide a uniform entry and exit speed and an injection speed and volume.

The number of cells administered to the subject can range from about 10²-10⁸ at each administration (e.g., injection site), depending on the size and species of the recipient, and the volume of tissue requiring cell replacement. Single administration (e.g., injection) doses can span ranges of 10³-10⁵, 10⁴-10⁷, and 10⁵-10⁸ cells, or any amount in total for a transplant recipient patient.

Since the CNS is an immunologically privileged site, administered cells, including xenogeneic, can survive and, optionally, no immunosuppressant drugs or a typical regimen of immunosuppressant agents are used in the treatment methods. However, optionally, an immunosuppressant agent may also be administered to the subject. Immunosuppressant agents and their dosing regimens are known to one of skill in the art and include such agents as Azathioprine, Azathioprine Sodium, Cyclosporine, Daltroban, Gusperimus Trihydrochloride, Sirolimus, and Tacrolimus. Dosages ranges and duration of the regimen can be varied with the disorder being treated; the extent of rejection; the activity of the specific immunosuppressant employed; the age, body weight, general health, sex and diet of the subject; the time of administration; the route of administration; the rate of excretion of the specific immunosuppressant employed; the duration and frequency of the treatment; and drugs used in combination. One of skill in the art can determine acceptable dosages for and duration of immunosuppression. The dosage regimen can be adjusted by the individual physician in the event of any contraindications or change in the subject's status.

### Recombinant Genetic Constructs

Another aspect of the present disclosure is directed to a recombinant genetic construct comprising: (i) a G-protein coupled receptor (GPR17) promoter-inclusive regulatory element sequence having a 5′ and a 3′ end and (ii) a nucleic acid sequence encoding a molecule of interest, wherein said nucleic acid sequence is heterologous to the GPR17 promoter-inclusive regulatory element and wherein said nucleic acid sequence is positioned 3′ to the GPR17 promoter-inclusive regulatory element sequence.

In some embodiments, the molecule of interest is selected from the group consisting of a therapeutic molecule, a post-transcriptional modulator of gene expression, a phenoconversion-promoting molecule, a gene-editing molecule, and an epigenetic editing molecule.

The terms “nucleic acid”, “nucleotide”, and “polynucleotide” encompass both DNA and RNA unless specified otherwise.

The “recombinant genetic constructs” of the disclosure are nucleic acid molecules containing a combination of two or more genetic elements not naturally occurring together. As described in more detail infra, the recombinant genetic construct may be expressed in a population of glial progenitor cells to induce expression of a therapeutic molecule, a post-transcriptional modulator of gene expression, a phenoconversion-promoting molecule, a gene-editing molecule, or an epigenetic editing molecule in the population of glial progenitor cells.

In any embodiment of the methods or recombinant genetic construct according to the present disclosure, the nucleic acid sequence encoding the therapeutic molecule, the post-transcriptional modulator of gene expression, the phenoconversion-promoting molecule, the gene-editing molecule, or the epigenetic editing molecule is operably linked to the GPR17 promoter-inclusive regulatory element. As used herein, the term “operably linked” describes the connection between regulatory elements and a nucleic acid sequence encoding the therapeutic molecule, the post-transcriptional modulator of gene expression, the phenoconversion-promoting molecule, the gene-editing molecule, or the epigenetic editing molecule according to the present disclosure. Typically, expression of the therapeutic molecule, the post-transcriptional modulator of gene expression, the phenoconversion-promoting molecule, the gene-editing molecule, or the epigenetic editing molecule is placed under the control of one or more regulatory elements, for example, without limitation, a promoter, a tissue-specific regulatory element, and/or an enhancer. A nucleic acid sequence encoding the therapeutic molecule, the post-transcriptional modulator of gene expression, the phenoconversion-promoting molecule, the gene-editing molecule, or the epigenetic editing molecule is said to be “operably linked to” the regulatory elements, meaning that the transcription of the nucleic acid sequence encoding the therapeutic molecule, the post-transcriptional modulator of gene expression, the phenoconversion-promoting molecule, the gene-editing molecule, or the epigenetic editing molecule is controlled or influenced by the regulatory element. For instance, a promoter is operably linked to a nucleic acid sequence encoding the therapeutic molecule, the post-transcriptional modulator of gene expression, the phenoconversion-promoting molecule, the gene-editing molecule, or the epigenetic editing molecule if the promoter effects transcription or expression of the coding sequence.

The term “regulatory element” refers to a nucleic acid sequence that can influence the expression of an operably linked coding sequence in a particular host organism. The term “regulatory element” is used broadly to cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873, which is hereby incorporated by reference in its entirety). Regulatory elements include, without limitation, transcriptional and translational control sequences, such as promoters, enhancers, splicing signals, polyadenylation signals, terminators, protein degradation signals, 2A sequences, and the like, that provide for and/or regulate expression of a coding sequence and/or production of an encoded polypeptide in a host cell.

The term “promoter” refers to a nucleotide sequence that permits binding of RNA polymerase and directs the transcription of a gene or a nucleic acid sequence encoding the therapeutic molecule, the post-transcriptional modulator of gene expression, the phenoconversion-promoting molecule, the gene-editing molecule, or the epigenetic editing molecule of interest. Typically, a promoter is located in the 5′ non-coding region of a gene, proximal to the transcriptional start site of the gene. Sequence elements within promoters that function in the initiation of transcription are often characterized by consensus nucleotide sequences.

The term “enhancer” refers to a segment of DNA which contains sequences capable of providing enhanced transcription and in some instances can function independent of their orientation relative to another control sequence. Enhancers are cis-acting DNA sequences that can function cooperatively or additively with promoters and/or other enhancer elements. The term “promoter/enhancer” or “promoter-inclusive regulatory element” refers to a segment of DNA which contains sequences capable of providing both promoter and enhancer functions.

Applicant has identified a G protein-coupled receptor 17 (“GPR17”) promoter-inclusive regulatory element that permits cell-specific expression of an encoded therapeutic molecule, post-transcriptional modulator of gene expression, phenoconversion-promoting molecule, gene-editing molecule, or epigenetic editing molecule by human oligodendrocyte progenitor cells. As described infra, cells that express GPR17 are destined for oligodendrocyte fate, and GPR17 is not expressed by any other brain cell phenotype. By placing the GPR17 promoter-inclusive regulatory element 5′ to a nucleic acid sequence encoding a therapeutic molecule, a post-transcriptional modulator of gene expression, a phenoconversion-promoting molecule, a gene-editing molecule, or an epigenetic editing molecule of interest, and placing the resulting recombinant genetic construct into an expression vector, Applicant has found that human oligodendrocyte-biased glial progenitor cells can be specifically targeted, both in vitro and in vivo.

As used herein, the phrase “GPR17 promoter-inclusive regulatory element” refers to a nucleotide sequence that directs the glial progenitor cell-specific transcription of a nucleic acid sequence encoding a therapeutic molecule, a post-transcriptional modulator of gene expression, a phenoconversion-promoting molecule, a gene-editing molecule, or an epigenetic editing molecule of interest. The GPR17 promoter-inclusive regulatory element according to the present disclosure comprises a portion of the 5′ untranslated region of GPR17 (NCBI Gene ID: 2840, which is hereby incorporated by reference in its entirety). In some embodiments of the methods and recombinant genetic constructs according to the present disclosure, the GPR17 promoter-inclusive regulatory element comprises a 5′ untranslated region of Homo sapiens G protein-coupled receptor 17 (GPR17) comprising the nucleic acid sequence of SEQ ID NO: 1 below. GGATACGGAAGAGATCCAATCGCAGACCCAAGATCCCCACCCAGGTTATGGTGGGCAGACCCCAG ATGCCAGGGCCACCCATTCAGCATCCCTCCCTGGACCCCAGGACCTGCTACTGCTGGGTGTCTGG ACTCCATCCTGCACAGCACTGTGCTCCATCTGCCCTGGGGTGTCTCATCATCAGCTGTGTGCAGG GCAAGGGGCCCAAACAAAGGCCCAGCAGTCACTGGCTAAGCTGCCGACTGGCTCTCTGTGCCTCC CCAAGACCCTATGTGCCCAGCAGGGGGCAACAGCTCAGGGTCAGCTGACCGAATGCCTCGGTGAA TGAATGACTCTACAAGAGAGGAAGGGAGCCTCGGTGGGCATCATCTCCCCTCGACTACTGGCCAG AGCCCTGGCTCTTACACCCCAGCGACGGGAAGCAGTTGTGGCCTGTGGCTTCAGTCTTCATCACC ACAATCCCTGAAGCCCACCCTTGCCCAGACACCTGTGCCCCAGCCCCAACCCCAGGCCACCTCCT CAGCAGGTCTGGGGCTGAGCTGCCCCACCTGGCGCCTATGGCGGCCAGCCCATGCCCCCTGCGGT GCCTCTGTCCCAGACTCAGCATGTAGGCCCCATGACCCCACTCCACATTCTGGTGACTCCTCCTG AGCGTCAGGACAACACTCAACCCACGAGGAATTATTTCTGTCTCAAAGATGCAGGAATCAGCTCA ACGCCTCAAAACTCCATCACCACGGTCAATGCCCTTGAAGCCATCGACAGTGATCACCCCAATAA CAGAAGGTCTGTGAGCCCAGAAATGCCCTGCTCAGGGTGGTTAGCTTCAAGCCACCACCTTTCCA ACCAGCCTGGGCCAGTTCTTCCAGACAGCCGCCTGCGGGCACAACAGGAAAGAGACCTGCGCCCC GGCTCAGACACCTCACACCCAGCTGGCTCTCAGGCCAGACAAACTGGGAAGCCCATCTCTCTTGA AGGAAGTCCAGATGGGAAACAGCTTCTCAACAGACCAGATCACAGCATCAGATCTAAAGGTGGCC TTCAGAATTCTTTTTCAGGTTGAATTAGGATCAAATCTAAGAATTCTAAATTCAAAATGCAGCAG AAAAACAAAACACACACACACACGGAGCCTAAGTTCTGGAGTGACATGTGCTTGGGTTCAAATCC TGGCTCTGTTGCTTCCTACTGTTTGTTGATGGGTGAGTTTCTTCATTTGCCTGAGCCTCAGTTTC CTTGTCTGTAAAATGGGGCAATAATCCCAGCTGCACAGGGTGATGTGAAGAGACAAATTTAAGAC ACTGCCCCTTAAATGCTAGCCACATACATACAGTTTTCAATGTTTAAACAACAAAATGTAAAGTC TTTTGAAACCAGGAAGGGTGATTTGGTTTCCCATGTTGCTGGATGTATCATTTTCAGAAAGACAG AGAGAAATGAACTTTGTTCACTCAGTCTCAGAGGCGGCCGCCGGCAGCATTCAAAGGCACCCCAG CCCGGAGCCACCCCAGGGAGGAGCCCCAGGCCAGCGGTCAGATTCATGGGCTTCCGTGCAGAAGG GGAGCTGCACCGGCGAGCACCCGGCCTCTGAGCTGAGCCGCATCCTCACGGACAGGACAGCGCCC CATTATGAGGCTCCTGCAGCTGTTCCTCGCTCCAGATAAAGGCCATGATTTATTCTGTGTGCCCA AATGGGGCCTCATTATACAGGGCAGGACACAAGGACCCTACAGCAAGTGTCCTCAAAGAGTCGCC TCTCACTCCGTGAGCAAGACTCCTCGGCCTCCCACCCTCCGTTCACAGGCCCCCTCCGCCGTCTG CGGGCGCAGGCCTGGGAGCGCCGCCTGTTGCCATGACAGCCGGCCCCTCCCTGCCCCCCATCAGT AGGAAATCATCCCCTTCTGAAACGTCCTGTTGTGTCCCTCAGCTCCAGCCCAAGCCCCCCACCCA GCCCCCGCCTGCTCTGAGTCTCTGAGACAGTCACACACTCAGACTATGTGGCCAAGCTGGGGGCG GGGGGCATGGGCTAGGGACACACTAGAATATTCACGCTCCGGTGGCAGCAGCAGCAGCAGCCAGA GGAGCAGCCCGACACAACAAGGGACCCCTCAGGAATGAAGCAGCCTTTCAGGGCCAGAGGGGCTG TGGTCTCCCTTCCTCTCCTTAAATAGCCAGCGTTCCACCCACAGCGGCAAGGGC (SEQ ID NO: 1; GPR17 (2.2)). Thus, in some embodiments of the methods and recombinant genetic constructs according to the present disclosure, the GPR17 promoter-inclusive regulatory element has the sequence of SEQ ID NO: 1.

In some embodiments of the methods and recombinant genetic constructs according to the present disclosure, the GPR17 promoter-inclusive regulatory element is a human GPR17 promoter-inclusive regulatory element.

In some embodiments of the methods and recombinant genetic constructs according to the present disclosure, the GPR17 promoter-inclusive regulatory element comprises a portion of SEQ ID NO: 1. For example, the GPR17 promoter-inclusive regulatory element may comprise a continuous stretch of 1-100, 1-200, 1-300, 1-400, 1-500, 1-600, 1-700, 1-800, 1-900, 1-1,000, 1-1,100, 1-1,200, 1-1,300, 1-1,400, 1-1,500, 1-1,600, 1-1,700, 1-1,800, 1-1,900, 1-2,000, 1-2,100, or 1-2,198 of SEQ ID NO: 1.

In some embodiments of the methods and recombinant genetic constructs according to the present disclosure, the GPR17 promoter-inclusive regulatory element comprises a 5′ untranslated region of Homo sapiens G protein-coupled receptor 17 (GPR17) comprising the nucleic acid sequence of SEQ ID NO: 2 below. GTTGCTGGATGTATCATTTTCAGAAAGACAGAGAGAAATGAACTTTGTTCACTCAGTCTCAGAGG CGGCCGCCGGCAGCATTCAAAGGCACCCCAGCCCGGAGCCACCCCAGGGAGGAGCCCCAGGCCAG CGGTCAGATTCATGGGCTTCCGTGCAGAAGGGGAGCTGCACCGGCGAGCACCCGGCCTCTGAGCT GAGCCGCATCCTCACGGACAGGACAGCGCCCCATTATGAGGCTCCTGCAGCTGTTCCTCGCTCCA GATAAAGGCCATGATTTATTCTGTGTGCCCAAATGGGGCCTCATTATACAGGGCAGGACACAAGG ACCCTACAGCAAGTGTCCTCAAAGAGTCGCCTCTCACTCCGTGAGCAAGACTCCTCGGCCTCCCA CCCTCCGTTCACAGGCCCCCTCCGCCGTCTGCGGGCGCAGGCCTGGGAGCGCCGCCTGTTGCCAT GACAGCCGGCCCCTCCCTGCCCCCCATCAGTAGGAAATCATCCCCTTCTGAAACGTCCTGTTGTG TCCCTCAGCTCCAGCCCAAGCCCCCCACCCAGCCCCCGCCTGCTCTGAGTCTCTGAGACAGTCAC ACACTCAGACTATGTGGCCAAGCTGGGGGCGGGGGGCATGGGCTAGGGACACACTAGAATATTCA CGCTCCGGTGGCAGCAGCAGCAGCAGCCAGAGGAGCAGCCCGACACAACAAGGGACCCCTCAGGA ATGAAGCAGCCTTTCAGGGCCAGAGGGGCTGTGGTCTCCCTTCCTCTCCTTAAATAGCCAGCGTT CCACCCACAGCGGCAAGGG (SEQ ID NO: 2; GPR17(0.8)). Thus, in some embodiments of the methods and recombinant genetic constructs according to the present disclosure, the GPR17 promoter-inclusive regulatory element has the sequence of SEQ ID NO: 2.

In some embodiments of the methods and recombinant genetic constructs according to the present disclosure, the GPR17 promoter-inclusive regulatory element comprises a modified nucleic acid sequence of SEQ ID NO: 1 or SEQ ID NO: 2. The modified sequence may have at least 80% sequence identity to the nucleic acid sequence of SEQ ID NO: 1 or SEQ ID NO: 2. In some embodiments, the modified sequence has 81%, 82%, 83%, 84%, 85%, 86%, 87%, 88%, 89%, 90%, 91%, 92%, 93%, 94%, 95%, 96%, 97%, 98%, 99%, or more sequence identity with the nucleic acid sequence of SEQ ID NO: 1 or SEQ ID NO: 2. In some embodiments, the modified sequence contains a mutation that enhances transcription of the nucleic acid sequence encoding the therapeutic molecule, the post-transcriptional modulator of gene expression, the phenoconversion-promoting molecule, the gene-editing molecule, or the epigenetic editing molecule.

In some embodiments of the methods and recombinant genetic constructs according to the present disclosure, the recombinant genetic construct further comprises a glial fibrillary acidic protein (GFAP) promoter-inclusive regulatory element, where the GFAP promoter-inclusive regulatory element is positioned within the recombinant genetic construct between the GPR17 promoter-inclusive regulatory element sequence and the nucleic acid sequence encoding the therapeutic molecule. In accordance with such embodiments, the recombinant genetic construct may comprise the nucleic acid sequence of SEQ ID NO: 3 below. ACATATCCTGGTGTGGAGTAGGGGACGCTGCTCTGACAGAGGCTCGGGGGCCTGAGCTGGCTCTG TGAGCTGGGGAGGAGGCAGACAGCCAGGCCTTGTCTGCAAGCAGACCTGGCAGCATTGGGCTGGC CGCCCCCCAGGGCCTCCTCTTCATGCCCAGTGAATGACTCACCTTGGCACAGACACAATGTTCGG GGTGGGCACAGTGCCTGCTTCCCGCCGCACCCCAGCCCCCCTCAAATGCCTTCCGAGAAGCCCAT TGAGCAGGGGGCTTGCATTGCACCCCAGCCTGACAGCCTGGCATCTTGGGATAAAAGCAGCACAG CCCCCTAGGGGCTGCCCTTGCTGTGTGGCGCCACCGGCGGTGGAGAACAAGGCTCTATTCAGCCT GTGCCCAGGAAAGGGGATCAGGGGATGCCCAGGCATGGACAGTGGGTGGCAGGGGGGGAGAGGAG GGCTGTCTGCTTCCCAGAAGTCCAAGGACACAAATGGGTGAGGGGAGAGCTCTCCCCATAGCTGG GCTGCGGCCCAACCCCACCCCCTCAGGCTATGCCAGGGGGTGTTGCCAGGGGCACCCGGGCATCG CCAGTCTAGCCCACTCCTTCATAAAGCCCTCGCATCCCAGGAGCGAGCAGAGCCAGAGCAGGTTG GAGAGGAGACGCATCACCTCCGCTGCTCGCGG (SEQ ID NO: 3; GFAP). Thus, in some embodiments of the methods and recombinant genetic constructs according to the present disclosure, the GPR17 promoter-inclusive regulatory element has the sequence of SEQ ID NO: 3.

In accordance with such embodiments, the recombinant genetic construct may comprise the nucleic acid sequence of SEQ ID NO: 4 below.

(SEQ ID NO: 4)
GGATACGGAAGAGATCCAATCGCAGACCCAAGATCCCCACCCAGGTTATGGTGGGCAGACCCCAG
ATGCCAGGGCCACCCATTCAGCATCCCTCCCTGGACCCCAGGACCTGCTACTGCTGGGTGTCTGG
ACTCCATCCTGCACAGCACTGTGCTCCATCTGCCCTGGGGTGTCTCATCATCAGCTGTGTGCAGG
GCAAGGGGCCCAAACAAAGGCCCAGCAGTCACTGGCTAAGCTGCCGACTGGCTCTCTGTGCCTCC
CCAAGACCCTATGTGCCCAGCAGGGGGCAACAGCTCAGGGTCAGCTGACCGAATGCCTCGGTGAA
TGAATGACTCTACAAGAGAGGAAGGGAGCCTCGGTGGGCATCATCTCCCCTCGACTACTGGCCAG
AGCCCTGGCTCTTACACCCCAGCGACGGGAAGCAGTTGTGGCCTGTGGCTTCAGTCTTCATCACC
ACAATCCCTGAAGCCCACCCTTGCCCAGACACCTGTGCCCCAGCCCCAACCCCAGGCCACCTCCT
CAGCAGGTCTGGGGCTGAGCTGCCCCACCTGGCGCCTATGGCGGCCAGCCCATGCCCCCTGCGGT
GCCTCTGTCCCAGACTCAGCATGTAGGCCCCATGACCCCACTCCACATTCTGGTGACTCCTCCTG
AGCGTCAGGACAACACTCAACCCACGAGGAATTATTTCTGTCTCAAAGATGCAGGAATCAGCTCA
ACGCCTCAAAACTCCATCACCACGGTCAATGCCCTTGAAGCCATCGACAGTGATCACCCCAATAA
CAGAAGGTCTGTGAGCCCAGAAATGCCCTGCTCAGGGTGGTTAGCTTCAAGCCACCACCTTTCCA
ACCAGCCTGGGCCAGTTCTTCCAGACAGCCGCCTGCGGGCACAACAGGAAAGAGACCTGCGCCCC
GGCTCAGACACCTCACACCCAGCTGGCTCTCAGGCCAGACAAACTGGGAAGCCCATCTCTCTTGA
AGGAAGTCCAGATGGGAAACAGCTTCTCAACAGACCAGATCACAGCATCAGATCTAAAGGTGGCC
TTCAGAATTCTTTTTCAGGTTGAATTAGGATCAAATCTAAGAATTCTAAATTCAAAATGCAGCAG
AAAAACAAAACACACACACACACGGAGCCTAAGTTCTGGAGTGACATGTGCTTGGGTTCAAATCC
TGGCTCTGTTGCTTCCTACTGTTTGTTGATGGGTGAGTTTCTTCATTTGCCTGAGCCTCAGTTTC
CTTGTCTGTAAAATGGGGCAATAATCCCAGCTGCACAGGGTGATGTGAAGAGACAAATTTAAGAC
ACTGCCCCTTAAATGCTAGCCACATACATACAGTTTTCAATGTTTAAACAACAAAATGTAAAGTC
TTTTGAAACCAGGAAGGGTGATTTGGTTTCCCATGTTGCTGGATGTATCATTTTCAGAAAGACAG
AGAGAAATGAACTTTGTTCACTCAGTCTCAGAGGCGGCCGCCGGCAGCATTCAAAGGCACCCCAG
CCCGGAGCCACCCCAGGGAGGAGCCCCAGGCCAGCGGTCAGATTCATGGGCTTCCGTGCAGAAGG
GGAGCTGCACCGGCGAGCACCCGGCCTCTGAGCTGAGCCGCATCCTCACGGACAGGACAGCGCCC
CATTATGAGGCTCCTGCAGCTGTTCCTCGCTCCAGATAAAGGCCATGATTTATTCTGTGTGCCCA
AATGGGGCCTCATTATACAGGGCAGGACACAAGGACCCTACAGCAAGTGTCCTCAAAGAGTCGCC
TCTCACTCCGTGAGCAAGACTCCTCGGCCTCCCACCCTCCGTTCACAGGCCCCCTCCGCCGTCTG
CGGGCGCAGGCCTGGGAGCGCCGCCTGTTGCCATGACAGCCGGCCCCTCCCTGCCCCCCATCAGT
AGGAAATCATCCCCTTCTGAAACGTCCTGTTGTGTCCCTCAGCTCCAGCCCAAGCCCCCCACCCA
GCCCCCGCCTGCTCTGAGTCTCTGAGACAGTCACACACTCAGACTATGTGGCCAAGCTGGGGGCG
GGGGGCATGGGCTAGGGACACACTAGAATATTCACGCTCCGGTGGCAGCAGCAGCAGCAGCCAGA
GGAGCAGCCCGACACAACAAGGGACCCCTCAGGAATGAAGCAGCCTTTCAGGGCCAGAGGGGCTG
TGGTCTCCCTTCCTCTCCTTAAATAGCCAGCGTTCCACCCACAGCGGCAAGGGCGCGCCGGAGTC
GCTGCGCGCTGCCTTCGCCCCGTGCCCCGCTCCGCCGtcgacatatcctggtgtggagtagggga
cgctgctctgacagaggctcgggggcctgagctggctctgtgagctggggaggaggcagacagcc
aggccttgtctgcaagcagacctggcagcattgggctggccgccccccagggcctcctcttcatg
cccagtgaatgactcaccttggcacagacacaatgttcggggtgggcacagtgcctgcttcccgc
cgcaccccagcccccctcaaatgccttccgagaagcccattgagcagggggcttgcattgcaccc
cagcctgacagcctggcatcttgggataaaagcagcacagccccctaggggctgcccttgctgtg
tggcgccaccggcggtggagaacaaggctctattcagcctgtgcccaggaaaggggatcagggga
tgcccaggcatggacagtgggtggcagggggggagaggagggctgtctgcttcccagaagtccaa
ggacacaaatgggtgaggggagagctctccccatagctgggctgcggcccaaccccaccccctca
ggctatgccagggggtgttgccaggggcacccgggcatcgccagtctagcccactccttcataaa
gccctcgcatcccaggagcgagcagagccagagcaggttggagaggagacgcatcacctccgctg
ctcgcggTCGACCCGCCTCGCGCCGCCCGCCCCGGCTCTGACTGACCGCGTTACTCCCACAGGTG
AGCGGGCGGGACGGCCCTTCTCCTCCGGGCTGTAATTAGCTGAGCAAGAGGTAAGGGTTTAAGGG
ATGGTTGGTTGGTGGGGTATTAATGTTTAATTACCTGGAGCACCTGCCTGAAATCACTTTTTTTC
AG;

Uppercase text indicates the sequence of the GPR17 (2.2) promoter; bold uppercase text indicates intron sequences; lowercase text indicates the GFAP promoter). Thus, in some embodiments of the methods and recombinant genetic constructs according to the present disclosure, the GPR17 promoter-inclusive regulatory element has the sequence of SEQ ID NO: 4.

In some embodiments of the methods and recombinant genetic constructs according to the present disclosure, the molecule of interest is a therapeutic molecule. Accordingly, the nucleic acid sequence encodes a therapeutic molecule. The therapeutic molecule may be a polypeptide or nucleic acid molecule. The term “polypeptide,” “peptide” or “protein” are used interchangeably and to refer to a polymer of amino acid residues. The terms encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The therapeutic molecule may be a transcription factor. The term “transcription factor” refers to a DNA-binding protein that regulates the expression of a specific genes. In some embodiments, when the transcription factor has a positive effect on gene transcription, the transcription factor is a “transcription activation factor” or “activator.” The term “activator” generally refers to any protein that binds to DNA and thus regulates the expression of a nucleic acid molecule by increasing its rate of transcription.

The transcription factor may be selected from the group consisting of TCF7L2, PDGFRA, ZNF488, OLIG2, CSPG4, and SOX10. Exemplary nucleic acid sequences encoding a transcription factor of the present disclosure and amino acid sequences of the transcription factors of the present disclosure are set forth in Table 1 below.

TABLE 1
Exemplary Transcription Factor Sequences

| Transcription Factor | NCBI Ref. Sequence* |
|---|---|
| Homo sapiens transcription factor 7 like 2 (TCF7L2), | NM_001146274.2 |
| transcript variant 1, mRNA | NP_001139746.1 |
| Homo sapiens platelet derived growth factor receptor | NM_006206.6 |
| alpha (PDGFRA), transcript variant 1, mRNA | NP_006197.1 |
| Homo sapiens zinc finger protein 488 (ZNF488), | NM_153034.4 |
| transcript variant 2, mRNA | NP_694579.1 |
| Homo sapiens oligodendrocyte transcription factor 2 | NM_005806.4 |
| (OLIG2), mRNA | NP_005797.1 |
| Homo sapiens chondroitin sulfate proteoglycan 4 | NM_001897.5 |
| (CSPG4), mRNA | NP_001888.2 |
| Homo sapiens SRY-box transcription factor 10 | NM_006941.4 |
| (SOX10), mRNA | NP_008872.1 |

*Each of which is hereby incorporated by reference in its entirety.

In some embodiments, the nucleic acid sequences encoding a transcription factor of the present disclosure comprises a portion, variant, or modified sequence of any of the nucleic acid sequences identified in Table 1 above. Thus, in some embodiments, the nucleic acid sequence encodes a transcription factor having an amino acid sequence, where the amino acid sequence has at least 80% sequence identity (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity) to any of the amino acid sequences provided in Table 1.

In some embodiments, the therapeutic molecule decreases, suppresses, attenuates, diminishes, arrests, or stabilizes the development or progression of a neurodegenerative disease, disorder, or condition in a host organism.

In some embodiments of the methods and recombinant genetic constructs according to the present disclosure, the molecule of interest is a post-transcriptional modulator of gene expression. In accordance with such embodiments, the nucleic acid sequence encodes a post-transcriptional modulator of gene expression. Modulation of gene expression as described herein can be carried out using antisense approaches which involve the design of oligonucleotides (either DNA, RNA, DNA/RNA, or chemically modified derivatives thereof) that are complementary to an RNA encoded by polynucleotide sequences of the genes identified herein. Antisense RNA may be introduced into a cell to inhibit translation or activity of a complementary mRNA by base pairing to it and physically obstructing its translation or its activity. This effect is therefore stoichiometric. Absolute complementarity, although preferred, is not required. A sequence “complementary” to a portion of an RNA, as referred to herein, may refer to a sequence having sufficient complementarity to be able to hybridize with the RNA, forming a stable duplex. In the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex.

Thus, in some embodiments, the post-transcriptional modulator of gene expression is selected from the group consisting of antisense oligonucleotide (ASO), small interfering RNA (siRNA), short or small hairpin RNA (shRNA), and micro(RNA).

Suitable antisense oligonucleotides (ASOs) for use in the recombinant genetic constructs as described herein include, without limitation, antisense RNAs, DNAs, RNA/DNA hybrids (e.g., gapmer), and chemical analogues thereof, e.g., morpholinos, peptide nucleic acid oligomer, ASOs comprised of locked nucleic acids. With the exception of RNA oligomers, PNAs, and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack.

In some embodiments, the post-transcriptional modulator is an antisense oligonucleotide that specifically binds to and inhibits the functional expression of one or more genes described herein. The antisense oligonucleotide may comprise modification to increase duplex stability including, e.g., the incorporation of 5-methyl-dC, 2-amino-dA, locked nucleic acid, and/or peptide nucleic acid bases. Common modifications to enhance antisense oligonucleotide nuclease resistance include, e.g., conversion of normal phosphodiester linkages to phosphorothioate or phosphorodithioate linkages, or use of propyne analog bases, 2′-O-Methyl or 2′-O-Methyloxyethyl RNA bases.

In some embodiments, the post-transcriptional modulator is a small interfering RNA (siRNA). siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule. siRNA molecules are typically designed to target a region of the mRNA target approximately 50-100 nucleotides downstream from the start codon. The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. Upon introduction into a cell, the siRNA complex triggers the endogenous RNAi pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

In some embodiments, the post-transcriptional modulator is a short or small hairpin RNA (shRNA). Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway.

In some embodiments, the post-transcriptional modulator is a microRNA (miRNA). As used herein, the term “microRNA” or “miRNA” refers to a class of small RNA molecules that may negatively regulate gene expression (see, e.g., Lam et al., “siRNA Versus miRNA as Therapeutics for Gene Silencing,” Mol. Ther. Nucleic Acids 4(9):e252 (2015), which is hereby incorporated by reference in its entirety). miRNA gene transcription is carried out by RNA polymerase II in the nucleus to give primary miRNA (pri-miRNA), which is a 5′ capped, 3′ polyadenylated RNA with double-stranded stem-loop structure. The pri-miRNA is then cleaved by a microprocessor complex (comprising Drosha and microprocessor complex subunit DCGR8) to form precursor miRNA (pre-miRNA), which is a duplex that contains 70-100 nucleotides with interspersed mismatches and adopts a loop structure. The pre-miRNA is subsequently transported by Exportin 5 from the nucleus to the cytoplasm, where it is further processed by Dicer into a miRNA duplex of 18-25 nucleotides. The miRNA duplex then associates with the RISC forming a complex called miRISC. The miRNA duplex is unwound, releasing and discarding the passenger strand (sense strand). The mature single-stranded miRNA guides the miRISC to the target mRNAs. Mature miRNA may bind to a target mRNA through partial complementary base pairing with the consequence that the target gene silencing occurs via translational repression, degradation, and/or cleavage.

miRNAs suitable for use in the recombinant genetic constructs disclosed herein include, without limitation, hsa-mir-9 (hsa-mir-9-5p) (miRBase Accession No. MIMAT0000441, which is hereby incorporated by reference in its entirety); hsa-mir-9 (hsa-mir-9-3p) (miRBase Accession No. MIMAT0000442, which is hereby incorporated by reference in its entirety); hsa-mir-124-1 (miRBase Accession No. MI0000443, which is hereby incorporated by reference in its entirety); hsa-mir-124-2 (miRBase Accession No. MI0000444, which is hereby incorporated by reference in its entirety); and hsa-mir-124-3 (miRBase Accession No. MI0000445, which is hereby incorporated by reference in its entirety) (see, e.g., Nowek et al., “The Versatile Nature of miR-9/9* in Human Cancer,” Oncotarget. 9(29):20838-20854 (2018), which is hereby incorporated by reference in its entirety).

miR-9/9* and miR-124 are essential for neuronal differentiation and the maintenance of neuronal identity through the repression of anti-neural genes including cofactors of the REST complex, RCOR1, and SCP1 (see, e.g., Lu and Yoo, “Mechanistic Insights Into MicroRNA-Induced Neuronal Reprogramming of Human Adult Fibroblasts,” Front. Neurosci. 12:522 (2018), which is hereby incorporated by reference in its entirety). miR-9 (miR-9-5p) and miR-9* (miR-9-3p) are two miRNAs that originate from the same precursor and are highly conserved during evolution from flies to humans (see Nowek et al., “The Versatile Nature of miR-9/9* in Human Cancer,” Oncotarget 9(29): 20838-20854 (2018), which is hereby incorporated by reference in its entirety). miR-124 represses translation of a large number of non-neuronal transcripts (Lim et al., “Microarray Analysis shows that some microRNAs Downregulate Large Numbers of Target mRNAs,” Nature 43: 769-773 (2005), which is hereby incorporated by reference in its entirety) and is a well-known regulator of the transcription silencing complex built on REST, which represses a large array of neuronal-specific genes in non-neuronal cells; this includes miR-124 itself, thus forming an auto-regulatory loop during neuronal differentiation (Xue et al., “Direct Conversion of Fibroblasts to Neurons by Reprogramming PTB-Regulated microRNA Circuits,” Cell 152(1-2): 82-96 (2013), which is hereby incorporated by reference in its entirety). Thus, in some embodiments, the one or more reprogramming factors is selected from, e.g., miR-9/9* and miR-124.

Methods of designing nucleic acid molecules are well known in the art and suitable for designing the post-transcriptional modulators of gene expression (e.g., ASOs, siRNAs, shRNAs, miRNAs, aptamers) described herein (see, e.g., Lam et al., “siRNA Versus miRNA as Therapeutics for Gene Silencing,” Mol. Ther. Nucleic Acids 4(9):e252 (2015) and Kulkarni et al., “The Current Landscape of Nucleic Acid Therapeutics,” Nature Nanotechnology 16:630-643 (2021), which are hereby incorporated by reference in their entirety).

In some embodiments of the methods and recombinant genetic constructs according to the present disclosure, the molecule of interest is a phenoconversion-promoting molecule. In accordance with such embodiments, nucleic acid sequence encodes a phenoconversion-promoting molecule. The phenoconversion-promoting molecule may comprise a neuronal reprogramming factor.

Suitable neuronal reprogramming factors include, without limitation, medium spiny neuron reprogramming factors, cortical interneuron reprogramming factors, dopaminergic neuron reprogramming factors, peripheral sensory neuron reprogramming factors, nonadrenergic neuronal reprogramming factors, cholinergic reprogramming factors, and spinal motor neuron reprogramming factors. Exemplary neuronal reprogramming factors are described in more detail infra.

Without being bound by theory, expression of one or more of the neuronal reprogramming factors disclosed herein in a population of glial progenitor cells may be effective to generate a population of neurons (e.g., striatal medium spiny neurons or dopaminergic nigrostriatal neurons) suitable for the treatment of neurodegenerative disorders. Accordingly, the recombinant genetic construct according to the present disclosure may comprise a nucleic acid sequence encoding one or more neuronal reprogramming factors selected from the group consisting of a microRNA (e.g., miR-9/9*, miR-124), an inhibitor of polypyrimidine-tract-binding protein 1 (PTBP1), an inhibitor of repressor element-1 (RE1) silencing transcription factor (REST), and one or more transcription factors.

As described herein, regulated RNA processing plays a critical role in neuronal differentiation. The polypyrimidine tract binding protein PTB and its homolog nPTB undergo a programmed switch during neuronal differentiation. miR-124 is able to modulate such switch by reducing PTB, thereby reprogramming an array of neuronal-specific alternative splicing events and forced expression of PTB is able to block miR-124 induced neuronal differentiation (Xue et al., “Direct Conversion of Fibroblasts to Neurons by Reprogramming PTB-Regulated microRNA Circuits,” Cell 152(1-2):82-96 (2013), which is hereby incorporated by reference in its entirety). Thus, in some embodiments, the one or more neuronal reprogramming factors is an inhibitor of polypyrimidine-tract-binding protein 1 (PTBP1; PTB).

The inhibitor of PTBP1 may be any antisense oligonucleotide (ASO), small interfering RNA (siRNA), short or small hairpin RNA (shRNA), and microRNA (miRNA) which reduces or eliminates the expression of PTB.

As used herein, the term “reduce” or “reduces” refers to its meaning as is generally accepted in the art. The term “reduce” or “reduces” generally refers to a suppression in the transcription and/or translation of a gene (e.g., PTBP1) or in the levels of the gene product relative to the transcription and/or translation of the gene observed in the absence of the nucleic acid inhibitor. In some embodiments, the reduction in the transcription and/or translation of a gene or in the levels of the gene product is at least 10%, at least 20%, at least 30%, at least 40%, at least 50%, at least 60%, at least 70%, at least 80%, at least 90%, at least 95%, at least 96%, at least 97%, at least 98%, at least 99%, up to 100% (i.e., no detectable transcription and/or translation) or a reduction of at least 2-fold, at least 5-fold, at least 10-fold, at least 20-fold, at least 30-fold, at least 40-fold, at least 50-fold, or more relative to that observed in the absence of the nucleic acid inhibitor molecule according to the present disclosure.

Suitable nucleic inhibitors of PTBP1 include, without limitation, a PTBP1 antisense oligonucleotide (ASO), a PTBP1 small interfering RNA (siRNA), a PTBP1 short or small hairpin RNA (shRNA), and a PTBP1 microRNA (miRNA). Such inhibitors may be designed in a sequence specific manner. The sequence of PTBP1 is well known in the art and accessible via various curated databases, e.g., NCBI nucleotide or gene database.

In some embodiments, the PTBP1 ASO, PTBP1 siRNA, PTBP1 shRNA, or PTBP1 miRNA is designed to target the sequence of PTBP1 transcript variant X1 mRNA (NCBI Reference Sequence: XM_005259597.2, which is hereby incorporated by reference in its entirety), or a portion thereof.

The transcriptional repressor element-1 (RE1) silencing transcription factor (REST)/neuron-restrictive silencer factor (NRSF) is a gene silencing transcription factor that is widely expressed during embryogenesis and is critical to elaboration of the neuronal phenotype (Noh et al., “Repressor Element-1 Silencing Transcription Factor (REST)-Dependent Epigenetic Remodeling is Critical to Ischemia-Induced Neuronal Death,” PNAS 16:E962-E971 (2012), which is hereby incorporated by reference in its entirety). REST binds Neuron Restrictive Silencer Elements (NRSEs) in >2000 neuronal genes and represses their expression (Conaco et al., “Reciprocal Actions of REST and a microRNA Promote Neuronal Identity,” PNAS 103(7):2422-2427 (2006) and Schoenherr & Anderson, “The Neuron-Restrictive Silencer Factor (NRSF): A Coordinate Repressor of Multiple Neuron-Specific Genes,” Science 267:1360-1363 (1995), which are hereby incorporated by reference in their entirety). In some embodiments, the one or more neuronal reprogramming factors is an inhibitor of RE1-silencing transcription factor (REST).

The inhibitor of REST may be any antisense oligonucleotide (ASO), small interfering RNA (siRNA), short or small hairpin RNA (shRNA), and microRNA (miRNA) which reduces or eliminates the expression of REST. Such inhibitors may be designed in a sequence specific manner. The sequence of REST is well known in the art and accessible via various curated databases, e.g., NCBI nucleotide or gene database.

In some embodiments, the REST ASO, REST siRNA, REST shRNA, or REST miRNA is designed to target the sequence of Homo sapiens RE1 silencing transcription factor (REST), transcript variant 2, mRNA (NCBI Reference Sequence: NM_001193508.1 which is hereby incorporated by reference in its entirety), or a portion thereof.

In some embodiments, the REST ASO, REST siRNA, REST shRNA, or REST miRNA is designed to target the sequence of Homo sapiens RE1 silencing transcription factor (REST), transcript variant 3, mRNA (NCBI Reference Sequence: NM_001363453.2, which is hereby incorporated by reference in its entirety), or a portion thereof.

In some embodiments, the REST ASO, REST siRNA, REST shRNA, or REST miRNA is designed to target the sequence of Homo sapiens RE1 silencing transcription factor (REST), transcript variant 1, mRNA (NCBI Reference Sequence: NM_005612.5, which is hereby incorporated by reference in its entirety), or a portion thereof.

In some embodiments, the inhibitor of REST comprises a REST guide RNA and Cas protein (or a nucleic acid molecule encoding the Cas protein). Suitable Cas proteins and nucleic acid molecules encoding said Cas proteins are described in detail supra.

In some embodiments, the one or more neuronal reprogramming factors comprises one or more transcription factors. In some embodiments, the transcription factor is selected from CTIP2, DLX1, DLX2, MYT1L, FOXP1, FOXP2, ZFP503, RARB, RXRG, GSH2, ASCL1, BRN2, ZIC1, OLIG2, NGN2, NURR1, LMX1A, SOX2, NEUROD1, NEUROD2, ISL1, and LHX3.

Nucleic acid sequences and molecules encoding the one or more transcription factors identified herein are well known and accessible in the art. Exemplary nucleic acid sequences encoding a transcription factor of the present disclosure and amino acid sequences of the transcription factors of the present disclosure are set forth in Table 2 below.

TABLE 2
Exemplary Transcription Factor Sequences

| Transcription Factor | NCBI Reference. Sequence* |
|---|---|
| CTIP (Homo sapiens BAF chromatin remodeling complex | NM_001282237.2 |
| subunit BCL11B (BCL11B), transcript variant 3, mRNA) | NP_001269166.1 |
| Homo sapiens distal-less homeobox 1 (DLX1), transcript | NM_178120.5 |
| variant 1, mRNA | NP_835221.2 |
| Homo sapiens distal-less homeobox 2 (DLX2), mRNA | NM_004405.4 |
| | NP_004396.1 |
| Homo sapiens myelin transcription factor 1 like (MYT1L), | NM_001303052.2 |
| transcript variant 1, mRNA | NP_001289981.1 |
| Homo sapiens forkhead box P1 (FOXP1), transcript variant | NM_001349338.3 |
| 11, mRNA | NP_001336267.1 |
| Homo sapiens forkhead box P2 (FOXP2), transcript variant | NM_014491.4 |
| 1, mRNA | NP_055306.1 |
| Mus musculus zinc finger protein 503 (Zfp503), mRNA | NM_145459.3 |
| | NP_663434.2 |
| Homo sapiens retinoic acid receptor beta (RARB), | NM_000965.5 |
| transcript variant 1, mRNA | NP_000956.2 |
| Homo sapiens retinoid X receptor gamma (RXRG), | NM_006917.5 |
| transcript variant 1, mRNA | NP_008848.1 |
| Homo sapiens homeobox protein GSH-2 (GSH2) gene, | AF439445.1 |
| complete cds | AAM08285.1 |
| Homo sapiens achaete-scute family bHLH transcription | NM_004316.4 |
| factor 1 (ASCL1), mRNA | NP_004307.2 |
| BRN2 (Homo sapiens) POU class 3 homeobox 2 | NM_005604.4 |
| (POU3F2), mRNA | NP_005595.2 |
| Homo sapiens Zic family member 1 (ZIC1), mRNA | NM_003412.4 |
| | NP_003403.2 |
| Homo sapiens oligodendrocyte transcription factor 2 | NM_005806.4 |
| (OLIG2), mRNA | NP_005797.1 |
| NGN2 (Homo sapiens neurogenin 2 (NEUROG2), mRNA) | NM_024019.4 |
| | NP_076924.1 |
| NURR1 (Homo sapiens Nurr1 gene, complete cds) | AB017586.1 |
| | BAA75666.1 |
| Homo sapiens LIM homeobox transcription factor 1 alpha | NM_177398.4 |
| (LMX1A), transcript variant 1, mRNA | NP_796372.1 |
| Homo sapiens SRY-box transcription factor 2 (SOX2), | NM_003106.4 |
| mRNA | NP_003097.1 |
| Homo sapiens neuronal differentiation 1 (NEUROD1), | NM_002500.5 |
| transcript variant 1, mRNA | NP_002491.3 |
| Homo sapiens neuronal differentiation 2 (NEUROD2), | NM_006160.4 |
| mRNA | NP_006151.3 |
| Homo sapiens ISL LIM homeobox 1 (ISL1), mRNA | NM_002202.3 |
| | NP_002193.2 |
| Homo sapiens LIM homeobox 3 (LHX3), transcript variant | NM_178138.6 |
| 1, mRNA | NP_835258.1 |

*Each of which is hereby incorporated by reference in its entirety.

In some of the methods and recombinant genetic constructs according to the present disclosure, the nucleic acid sequences encoding a transcription factor of the present disclosure comprises a portion, variant, or modified sequence of any of the amino acid sequences identified in Table 2 above.

In any embodiment of the methods and recombinant genetic constructs disclosed herein, the one or more transcription factors may comprise CTIP2, DLX1, DLX2, MYT1L, or a combination thereof. In accordance with such embodiments, the one or more neuronal reprogramming factors comprise miR-9/9*, miR-124, CTIP2, DLX1, DLX2, MYT1L, or a combination thereof. Expression of miR-9/9* and miR-124 (miR-9/9*-124) together with BCL11B (also known as CTIP2), DLX1, DLX2, and MYT1L has been shown to guide the conversion of human postnatal and adult fibroblasts into an enriched population of neurons analogous to striatal medium spiny neurons (MSNs) (Victor et al., “Generation of Human Striatal Neurons by microRNA-Dependent Direct Conversion of Fibroblasts,” Neuron 84(2):311-323 (2014), which is hereby incorporated by reference in its entirety).

In any embodiment of the methods and recombinant genetic constructs disclosed herein, the one or more transcription factors comprise ASCL1, BRN2, MYTL1, or a combination thereof. The combination of ASCL1, BRN2A, and MYT1 has been shown to reprogram mouse fibroblasts to functional neurons (Grealish et al., “Brain Repair and Reprogramming: The Route to Clinical Translation,” J. Internal Med 280:265-275 (2016), which is hereby incorporated by reference in its entirety).

In any embodiment of the methods and recombinant genetic constructs disclosed herein, the one or more transcription factors comprise ASCL1, NURR1, LMX1A, or a combination thereof. In accordance with such embodiments, the one or more neuronal reprogramming factors comprise a REST inhibitor, ASCL1, NURR1, LMX1A, or a combination thereof. The use of a short hairpin RNA against the RE1-silencing transcription factor (REST) complex, together with ACL1, LMX1A, and NURR1 (together referred to as ALN) has recently been shown to reprogram human glial progenitor cells into induced dopaminergic neurons, which at three weeks following transduction, expressed DA-related genes, including TH, SLC6A3 (DAT), FOXA2, LMX1A, and PITX3 (Nolbrant et al., “Direct Reprogramming of Human Fetal- and Stem Cell-Derived Glial Progenitor Cells into Midbrain Dopaminergic Neurons,” Stem Cell Reports 15(4):869-882 (2020), which is hereby incorporated by reference in its entirety). Addition of FOXA2 resulted in a higher endogenous expression of the midbrain dopaminergic genes LMX1A, EN1, and OTX2 (Nolbrant et al., “Direct Reprogramming of Human Fetal- and Stem Cell-Derived Glial Progenitor Cells into Midbrain Dopaminergic Neurons,” Stem Cell Reports 15(4):869-882 (2020), which is hereby incorporated by reference in its entirety). Thus, in some embodiments, the one or more neuronal reprogramming factors comprise a REST inhibitor, ASCL1, NURR1, LMX1A, FOXA2, or a combination thereof.

In any embodiment of the methods and recombinant genetic constructs disclosed herein, the one or more neuronal reprogramming factors comprise the transcription factors ISL1 and/or LHX3. In accordance with such embodiments, the one or more neuronal reprogramming factors comprise miR-9/9*, miR-124, ISL1, LHX3, or a combination thereof (e.g., miR-9/9*, miR-124, and ISL1 or miR-9/9*, miR-124, and LHX3). The use of the motor neuron transcription factors ISL1 and LHX3 in combination with miR-9/9* and miR-124 has been shown to mediate the conversion of human fibroblasts to motor neurons (see, e.g., U.S. Patent Application Publication No. 2002/0377885 to Yoo et al. and Lu & Yoo, “Mechanistic Insights Into MicroRNA-Induced Neuronal Reprogramming of Human Adult Fibroblasts,” Front. Neurosci. 12:522 (2018), which are hereby incorporated by reference in their entirety).

In any embodiment of the methods and recombinant genetic constructs disclosed herein, the one or more neuronal reprogramming factors comprise the transcription factors SOX2 alone or in combination with ASCL1. Expression of the transcription factor SOX2 alone or in combination with the transcription factor ASCL1, has been shown to induce the conversion of genetically fate-mapped NG2 glia into induced doublecortin* neurons in the adult mouse cerebral cortex (Heinrich et al., “Sox2-Mediated Conversion of NG2 Glia into Induced Neurons in the Injured Adult Cerebral Cortex,” Stem Cell Reports 3(6):2014, which is hereby incorporated by reference in its entirety).

In any embodiment of the methods and recombinant genetic constructs disclosed herein, the one or more neuronal reprogramming factors comprise the transcription factor NEUROD1. Studies have shown that in vivo retroviral expression of NEUROD1 mediates the direct reprogramming of NG2 cells into glutamatergic and GABAergeic neurons (Guo et al., “In Vivo Direct Reprogramming of Reactive Glial Cells into Functional Neurons After Brain Injury and in an Alzheimer's Disease Model,” Cell Stem Cell 14(2):188-202 (2014), which is hereby incorporated by reference in its entirety).

In some embodiments of the methods and recombinant genetic constructs according to the present disclosure, the molecule of interest is a gene-editing molecule. In accordance with such embodiments, the nucleic acid sequence encodes a gene-editing molecule. The gene-editing molecule may be selected from the group consisting of an RNA-guided nuclease or modified RNA-guided nucleases, a zinc finger nuclease, and a transcription activator-like effector nuclease (TALEN).

In some embodiments of the methods and recombinant genetic constructs according to the present disclosure, the gene-editing molecule is an RNA-guided nuclease or modified RNA-guided nuclease. Suitable RNA-guided nucleases include, without limitation, Clustered Regularly Interspaced Short Palindromic Repeat-associated (“Cas”) proteins, e.g., Cas9, Cas12a, and Cas12b. As described herein, Cas proteins form a ribonucleoprotein complex with a guide RNA, which guides the Cas protein to a target DNA sequence. The Cas protein may be a Cas nuclease (i.e., Cas proteins capable of introducing a double strand break at a target nucleic acid sequence) or a Cas nickase (i.e., Cas protein derivatives capable of introducing a single strand break at a target nucleic acid sequence).

In some embodiments of the methods and recombinant genetic constructs according to the present disclosure, the Cas protein is a Cas9 protein. As used herein, the term “Cas9 protein” or “Cas9” includes any of the recombinant or naturally-occurring forms of the CRISPR-associated protein 9 (Cas9) or variants or homologs thereof. In some embodiments, the variants or homologs have at least 90%, 95%, 96%, 97%, 98%, 99% or 100% amino acid sequence identity across the whole sequence or a portion of the sequence (e.g., a 50, 100, 150, or 200 continuous amino acid portion) compared to a naturally occurring Cas9 protein. In some embodiments, the Cas9 protein is substantially identical to the protein identified by the UniProt reference number Q99ZW2, G3ECR1, J7RUA5, AOQ5Y3, or J3F2B0 (which are hereby incorporated by reference in their entirety) or a variant or homolog having substantial identity thereto. In some embodiments, the Cas9 protein is selected from the group consisting of a Cas9 nuclease and a Cas9 nickases.

In some embodiments of the methods and recombinant genetic constructs according to the present disclosure, the Cas protein is a Cas12a protein. As used herein, the term “Cas12a protein” or “Cas12a” includes any of the recombinant or naturally-occurring forms of the CRISPR-associated protein 12 (Cas12a) or variants or homologs thereof. In some embodiments, the variants or homologs have at least 90%, 95%, 96%, 97%, 98%, 99% or 100% amino acid sequence identity across the whole sequence or a portion of the sequence (e.g., a 50, 100, 150, or 200 continuous amino acid portion) compared to a naturally occurring Cas12a protein. In some embodiments, the Cas12a protein is substantially identical to the protein identified by the UniProt reference number AOQ7Q2, U2UMQ6, A0A7C6JPC1, A0A7C9H0Z9, or A0A7J0AY55 (which are hereby incorporated by reference in their entirety) or a variant or homolog having substantial identity thereto. In some embodiments, the Cas 12a protein is selected from the group consisting of a Cas12a nuclease and a Cas12a nickase.

In some embodiments of the methods and recombinant genetic constructs according to the present disclosure, the Cas protein is a Cas12b protein. As used herein, the term “Cas12b protein” or “Cas12b” includes any of the recombinant or naturally-occurring forms of the CRISPR-associated protein 12 (Cas12b) or variants or homologs thereof. In some embodiments, the variants or homologs have at least 90%, 95%, 96%, 97%, 98%, 99% or 100% amino acid sequence identity across the whole sequence or a portion of the sequence (e.g., a 50, 100, 150, or 200 continuous amino acid portion) compared to a naturally occurring Cas12b protein. In some embodiments, the Cas12b protein is substantially identical to the protein identified by the UniProt reference number T0D7A2, A0A6I3SPI6, A0A6I7FUC4, A0A6N9TP17, A0A6M1UF64, A0A7Y8V748, A0A7X7KIS4, A0A7X8X2U5, or A0A7X8UMW7 (which are hereby incorporated by reference in their entirety) or a variant or homolog having substantial identity thereto. In some embodiments, the Cas 12b protein is selected from the group consisting of a Cas12b nuclease and a Cas12b nickase.

In some embodiments of the methods and recombinant genetic constructs according to the present disclosure, the molecule of interest is a guide RNA. In accordance with such embodiments, the recombinant genetic construct encodes a guide RNA. As used herein, the term “guide RNA” or “gRNA” refers to a ribonucleotide sequence capable of binding a nucleoprotein, thereby forming ribonucleoprotein complex. In accordance with the recombinant genetic constructs and methods of the present disclosure, the guide RNA comprises (i) a DNA-targeting sequence that is complementary to a target nucleic acid sequence of (e.g., an inhibitor of polypyrimidine-tract-binding protein 1 (PTBP1), an inhibitor of RE1-silencing transcription factor (REST), and/or one or more transcription factors selected from CTIP2, DLX1, DLX2, MYT1L, FOXP1, FOXP2, ZFP503, RARB, RXRG, GSH2, ASCL1, BRN2, ZIC1, OLIG2, NGN2, NURR1, LMX1A, SOX2, NEUROD1, NEUROD2, ISL1, and LHX3) and (ii) a binding sequence for the Cas protein (e.g., Cas9 nuclease, Cas9 nickase, Cas12a nuclease, and Cas12a nickase).

In some embodiments of the methods and recombinant genetic constructs according to the present disclosure, the guide RNA is a single guide RNA molecule (single RNA nucleic acid), which may include a “single-guide RNA” or “sgRNA”. In other embodiments, the nucleic acid of the present disclosure includes two RNA molecules (e.g., joined together via hybridization at the binding sequence). Thus, the term guide RNA is inclusive, referring both to two-molecule nucleic acids and to single molecule nucleic acids (e.g., sgRNAs).

In some embodiments of the methods and recombinant genetic constructs according to the present disclosure, the gRNA is 10, 20, 30, 40, 50, 60, 70, 80, 90, 100 or more nucleic acid residues in length. In some embodiments, the gRNA is from 10 to 30 nucleic acid residues in length. In some embodiments, the gRNA is 20 nucleic acid residues in length. In some embodiments, the length of the gRNA is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In some embodiments, the gRNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In some embodiments, the gRNA is from 10 to 15, 10 to 20, 10 to 30, 10 to 40, or 10 to 50 residues in length.

In some embodiments of the methods and recombinant genetic constructs according to the present disclosure, the gene-editing molecule is a zinc finger nuclease. A ZFN is an artificial endonuclease that comprises at least 1 zinc finger motif (e.g., at least 2, 3, 4, or 5 zinc finger motifs) fused to a nuclease domain (e.g., the cleavage domain of the FokI restriction enzyme). Zinc finger domains can be engineered to target desired DNA sequences, which enable zinc-finger nucleases to target unique sequence within a complex genome (Urnov et al., “Genome Editing with Engineered Zinc Finger Nucleases,” Nat. Rev. Genet. 11(9): 636-646 (2010), which is hereby incorporated by reference in its entirety). Hetero-dimerization of two individual ZFNs at their target nucleic acid sequences can result in cleavage of a target sequence. For example, two individual ZFNs may bind opposite strands of a target DNA sequence to induce a double-strand break in the target nucleic acid sequence. Methods of designing suitable ZFNs for inclusion in the systems of the presently claimed disclosure are well known in the art (see, e.g., Urnov et al., “Genome Editing with Engineered Zinc Finger Nucleases,” Nat. Rev. Genet. 11(9): 636-646 (2010); Gaj et al., “Targeted Gene Knockout by Direct Delivery of Zinc-Finger Nuclease Proteins,” Nat. Methods 9(8): 805-807 (2012); U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; and 6,479,626, which are hereby incorporated by reference in their entirety). In some embodiments, recombinant genetic construct encodes a first and a second molecule of interest. In accordance with such embodiments, the first and second molecule of interest are a first and second gene editing nuclease, respectively. For example, the first and second gene editing nucleases may be FokI nucleases. In accordance with such embodiments, the first and second DNA binding motifs are zinc finger motifs.

In some embodiments of the methods and recombinant genetic constructs according to the present disclosure, the gene-editing molecule is a transcription activator-like effector nuclease (TALEN) (Joung & Sander, “TALENs: A Widely Applicable Technology for Targeted Genome Editing,” Nat. Rev. Mol. Cell Biol. 14(1): 49-55 (2013), which is hereby incorporated by reference in its entirety). A TALEN is an engineered transcription activator-like effector nuclease that comprises a DNA-binding domain (i.e., a transcription activator like (TAL) motif) and a nuclease domain (e.g., a cleavage domain of the FokI restriction enzyme). The DNA-binding domain (i.e., the transcription activator like (TAL) motif) comprises a series of 33-35 amino acid repeat domains that each recognize a single base pair. Hetero-dimerization of two individual TALENs at a target nucleic acid sequence can result in cleavage of the target sequence. For example, two individual TALENs may bind opposite strands of a target DNA sequence to induce a double-strand break in the target nucleic acid sequence. Methods of designing suitable TALENs for inclusion in the systems of the presently claimed disclosure are well known in the art (see, e.g., Scharenberg et al., “Genome Engineering with TAL-Effector Nucleases and Alternative Modular Nuclease Technologies,” Curr. Gene Ther. 13(4): 291-303 (2013); Gaj et al., “Targeted Gene Knockout by Direct Delivery of Zinc-Finger Nuclease Proteins,” Nat. Methods 9(8):805-807 (2012); Beurdeley et al., “Compact Designer TALENs for Efficient Genome Engineering,” Nat. Commun. 4:1762 (2013); U.S. Pat. Nos. 8,440,431; 8,440,432; 8,450,471; 8,586,363; and 8,697,853, which are hereby incorporated by reference in their entirety). In some embodiments, when the recombinant genetic construct encodes a first and a second molecule of interest and the first and second molecule of interest are a first and second gene editing nuclease, the first and second gene editing nucleases may be FokI nucleases. In accordance with such embodiments, the first and second DNA binding motifs are transcription activator like (TAL) motifs.

In some embodiments of the methods and recombinant genetic constructs according to the present disclosure, the molecule of interest is an epigenic editing molecule. In accordance with such embodiments, the nucleic acid sequence encodes an epigenetic editing molecule. The epigenetic molecule may be selected from the group consisting of a DNA methyltransferase enzyme (e.g., DNA methyltransferase 3 alpha (DNMT3A) and DNA methyltransferase 3 like (DNMT3L)), a histone demethylation enzyme (e.g., lysine-specific histone demethylase 1 (LSD1)), a histone methyltransferase enzyme (e.g., G9A and SuV39h1), a transcription factor recruitment domain (e.g., Kruppel-associated box domain (KRAB), KRAB-Methyl-CpG binding protein 2 domain (KRAB-MeCP2), enhancer of Zeste 2 (EZH2)), and a zinc finger transcriptional repressor domain (e.g., spalt like transcription factor 1 (SALL1) and suppressor of defective silencing protein 3 (SDS3), G9A, and Suv39h1) (see, e.g., Brezgin et al., “Dead Cas Systems: Types, Principles, and Applications,” Int. J. Mol. Sci. 20:6041 (2019); Yeo et al., “An Enhanced CRISPR Repressor for Targeted Mammalian Gene Regulation,” Nat. Methods 15(8): 611-616 (2018); Alerasool et al., “An Efficient KRAB Domain for CRISPRi Applications in Human Cells,” Nature Methods 17(11):1093-1096 (2020); and Duke et al., “An Improved CRISPR/dCas9 Interference Tool for Neuronal Gene Suppression,” Frontiers in Genome Editing 2:9 (2020), which are hereby incorporated by reference in their entirety).

In some embodiments of the methods and recombinant genetic constructs according to the present disclosure, the epigenetic modulator is selected from the group consisting of Tet methylcytosine dioxygenase 1 (TET1), SunTag-TET1, MS2/MCP-TET1, p300Core, four tandem copies of herpes simplex viral protein 16 (VP64), VP160, NF-KB p65 activation domain (p65), Epstein-Barr Virus-derived R transactivator (Rta), SunTag-VP64, VP64-p65-Rta (VPR), SunTag-p65-HSF1, TV, synergistic activation mediator (SAM), Three-Component Repurposed Technology for Enhanced Expression (TREE), Casilio, Scaffold, and CMV (see, e.g., Brezgin et al., “Dead Cas Systems: Types, Principles, and Applications,” Int. J. Mol. Sci. 20(23):6041 (2019), which is hereby incorporated by reference in its entirety). In some embodiments, when demethylation of a gene or gene protein is effective to suppress its transcription, the epigenetic modulator is a demethylase (e.g., TET1).

In some embodiments of the methods and recombinant genetic constructs according to the present disclosure, epigenetic editing molecule mediates CRISPR interference. As used herein, the term “CRISPR interference” or “CRISPRi” refers to a system that allows for sequence-specific repression of gene expression. CRISPRi systems comprise nuclease dead Cas (“dCas”) proteins (i.e., nuclease-inactivated Cas proteins) to block the transcription of a target gene, without cutting the target DNA sequence. Nuclease inactivated Cas proteins and methods of generating nuclease-inactivated Cas proteins are well known in the art (see, e.g., Qi et al., “Repurposing CRISPR as an RNA-Guided Platform for Sequence-Specific Control of Gene Expression,” Cell 152(5): 1173-1183 (2013), which is hereby incorporated by reference in its entirety). Suitable nuclease dead Cas proteins include, e.g., dCas9, dCas12a, and dCas12b.

In some embodiments of the methods and recombinant genetic constructs according to the present disclosure, the nuclease dead Cas protein is a fusion protein comprising a Cas protein and one or more epigenetic modulators that suppress or silence the expression of the target gene, e.g., PTBP1.

In any of the methods and recombinant genetic constructs according to the present disclosure, the nuclease dead Cas protein is fused to a methyltransferase. In any embodiment, the nuclease dead Cas fusion protein is fused to a demethylase.

Suitable nuclease dead Cas fusion proteins are identified in Table 3 below.

TABLE 3
Exemplary Cas Fusion Proteins

| Fusion Protein | Reference* |
|---|---|
| dCas9-DNMT3A | Vojta et al., “Repurposing the CRISPR-Cas9 System for Targeted DNA |
| | Methylation,” Nucleic Acids Res. 44(12): 5615-5628 (2016) |
| dCas-SunTag- | Huang et al., “DNA Epigenome Editing using CRISPR-Cas SunTag- |
| DNMT3A | Directed DNMT3A,” Genome Biol. 18(1): 176 (2017) |
| dCas9-EZH2 | O'Geen et al., “Ezh2-dCas9 and KRAB-dCas9 Enable Engineering of |
| | Epigenetic Memory in a Context-Dependent Manner,” Epigenet. |
| | Chromatin. 12(1): 26 (2019) |
| dCas9-DNMT3A-3L | Brezgin et al., “Dead Cas Systems: Types, Principles, and |
| | Applications,” Int. J. Mol. Sci. 20(23): 6041 (2019) |
| dCas9-KRAB | O'Geen et al., “Ezh2-dCas9 and KRAB-dCas9 Enable Engineering of |
| | Epigenetic Memory in a Context-Dependent Manner,” Epigenet. |
| | Chromatin. 12(1): 26 (2019) |
| dCas9-KRAB-MeCP2 | Yeo et al., “An Enhanced CRISPR Repressor for Targeted Mammalian |
| | Gene Regulation,” Nat. Methods. 15(8): 611-616 (2018) |
| dCas12a-KRAB | Liu et al., “Engineering Cell Signaling Using Tunable CRISPR-Cpf1- |
| | based Transcription Factors,” Nat. Commun. 8: 2095 (2017) |
| dCas9-Chromo | Gilbert et al., “CRISPR-Mediated Modular RNA-Guided Regulation of |
| Shadow domain of | Transcription in Eukaryotes,” Cell 154: 442-451 (2013) |
| HP1α (“dCas9-Cs”) | |
| dCas9-WRPW domain | Gilbert et al., “CRISPR-Mediated Modular RNA-Guided Regulation of |
| of Hes1 (“dCas9- | Transcription in Eukaryotes,” Cell 154: 442-451 (2013) |
| WRPW”) | |
| dCas9-TET1 | Liu et al., “Editing DNA Methylation in the Mammalian Genome,” Cell |
| | 167: 233-247 (2016) |
| dCas9-SunTag-TET1 | Morita et al., “Targeted DNA Demethylation in vivo using dCas9- |
| | Peptide Repeat and scFv-TET1 Catalytic Domain Fusions,” Nat. |
| | Biotechnol. 34: 1060-1065 (2016) |
| dCas9-MS2/MCP- | Xu et al., “A CRISPR-Based Approach for Targeted DNA |
| TET1 | Demethylation,” Cell Discov. 2: 16009 (2016) |
| dCas9-P300 Core | Hilton et al., “Epigenome Editing by a CRISPR/Cas9-Based |
| | Acetyltransferase Activates Genes from Promoter and Enhancers,” Nat. |
| | Biotechnol. 33(5): 510-517 (2015) |
| dCas9-VP64 | Gilbert et al., “CRISPR-Mediated Modular RNA-Guided Regulation of |
| | Transcription in Eukaryotes,” Cell 154: 442-451 (2013) |
| dCas9-VP160 | Cheng et al., “Multiplexed Activators of Endogenous Genes by |
| | CRISPR-on, an RNA-Guided Transcriptional Activator System,” Cell |
| | Res. 23: 1163 (2013) |
| dCas9-p65 | Gilbert et al., “CRISPR-Mediated Modular RNA-Guided Regulation of |
| | Transcription in Eukaryotes,” Cell 154: 442-451 (2013) |
| dCas9-VPR | Chavez et al., “Highly Efficient Cas9-Mediated Transcriptional |
| | Programming,” Nat. Methods. 12: 326 (2015) |
| dCas9 SAM system | Konermann et al., “Genome-Scale Transcriptional Activation by an |
| | Engineered CRISPR-Cas9 Complex,” Nature 517(7536): 583-588 |
| | (2015) |
| dCas9 TREE system | Kunii et al., “Three-Component Repurposed Technology for Enhanced |
| | Expression: Highly Accumulable Transcriptional Activators via |
| | Branched Tag Arrays,” CRISPR J. 1(5): 337-347 (2018) |
| dCas9-TV (i.e., | Li et al., “A potent Cas9-Derived Gene Activator for Plant and |
| dCas9-6TAL-VP128) | Mammalian Cells,” Nat. Plants. 3: 930-936 (2017) |
| dCas12a-VPR | Liu et al., “Engineering Cell Signaling Using Tunable CRISPR-Cpf1- |
| | Based Transcription Factors,” Nat. Commun. 8: 2095 (2017) |
| dCas12a-p65 | Tak et al., “Inducible and Multiplex Gene Regulation Using CRISPR- |
| | Cpf1-Based Transcription Factors,” Nat. Methods 14: 1163-1166 (2017) |

*Each of which is hereby incorporated by reference in its entirety.

In some embodiments of the methods and recombinant genetic constructs according to the present disclosure, the nucleic acid sequence encodes a first molecule of interest and a second molecule of interest. In accordance with such embodiments, (i) the first molecule of interest is selected from the group consisting of a first nucleic sequence encoding a first therapeutic molecule, a first post-transcriptional modulator of gene expression, a first phenoconversion-promoting molecule, a first gene-editing molecule, or a first epigenetic editing molecule and (ii) the second molecule of interest is selected from the group consisting of a second nucleic acid sequence encoding a second therapeutic molecule, a second post-transcriptional modulator of gene expression, a second phenoconversion-promoting molecule, a second gene-editing molecule, or a second epigenetic editing molecule.

In some embodiments of the methods and recombinant genetic constructs according to the present disclosure, the nucleic acid molecule further encodes a reporter molecule. The reporter molecule may be selected from the group consisting of a fluorescent protein, a luminescent protein, and a fluorogenic nucleic acid aptamer.

In some embodiments of the methods and recombinant genetic constructs according to the present disclosure, the reporter molecule is a fluorescent protein. Suitable fluorescent proteins include, without limitation, green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, mRasberry, mStrawberry, Jred), and orange fluorescent proteins (mOrange, mKO, Kusabira-Orange, Monomeric Kusabira-Orange, mTangerine, tdTomato), or any other suitable fluorescent protein. In certain embodiments, the reporter protein is a fluorescent protein selected from the group consisting of green fluorescent protein (GFP), enhanced green fluorescent protein (EGFP), and yellow fluorescent protein (YFP).

In some embodiments of the methods and recombinant genetic constructs according to the present disclosure, the reporter molecule is a luminescent protein. Suitable luminescent proteins include luciferase. As used herein, the term “luciferase” refers to members of a class of enzymes that catalyze reactions that result in production of light. Luciferases have been identified in and cloned from a variety of organisms including fireflies, click beetles, sea pansy (Renilla), marine copepods, and bacteria among others. Examples of luciferases that may be used as reporter proteins include, e.g., Renilla (e.g., Renilla reniformis) luciferase, Gaussia (e.g., Gaussia princeps) luciferase), Metridia luciferase, firefly (e.g., Photinus pyralis luciferase), click beetle (e.g., Pyrearinus termitilluminans) luciferase, deep sea shrimp (e.g., Oplophorus gracilirostris) luciferase). Luciferase reporter proteins include both naturally occurring proteins and engineered variants designed to have one or more altered properties relative to the naturally occurring protein, such as increased photostability, increased pH stability, increased fluorescence or light output, reduced tendency to dimerize, oligomerize, aggregate or be toxic to cells, an altered emission spectrum, and/or altered substrate utilization.

In some embodiments of the methods and recombinant genetic constructs according to the present disclosure, the reporter molecule is a fluorogenic nucleic acid aptamer. Nucleic acid aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence. As used herein, the term “fluorogenic nucleic acid aptamer” refers to a nucleic acid aptamer (e.g., an RNA aptamer) that can bind and turn on an otherwise non-fluorescent small molecule dye (Filonov et al., “Broccoli: Rapid Selection of an RNA Mimic of Green Fluorescent Protein by Fluorescence-Based Selection and Directed Evolution,” J. Am. Chem. Soc. 136(46):16299-308 (2014), which is hereby incorporated by reference in its entirety). Suitable fluorogenic nucleic acid aptamers are well known in the art (see, e.g., Li et al., “Fluorophore-Promoted RNA Folding and Photostability Enables Imaging of Single Broccoli-Tagged mRNAs in Live Mammalian Cells,” Angew Chem. Int. Ed. Engl. 59(11):4511-4518 (2020) and U.S. Pat. No. 10,444,224 to Jaffrey et al., which are hereby incorporated by reference in their entirety).

In some embodiments of the methods and recombinant genetic constructs according to the present disclosure, the first and second molecules of interest are each polypeptides. In accordance with such embodiments, the first post-transcriptional modulator of gene expression, the first phenoconversion-promoting molecule, the first gene-editing molecule, or the first epigenetic editing molecule and the second therapeutic molecule, the second post-transcriptional modulator of gene expression, the second phenoconversion-promoting molecule, the second gene-editing molecule, or the second epigenetic editing molecule are each polypeptides.

In accordance with such embodiments, the nucleic acid sequence encoding the therapeutic molecule, the post-transcriptional modulator of gene expression, the phenoconversion-promoting molecule, the gene-editing molecule, and/or the epigenetic editing molecule further comprises: a self-cleaving peptide encoding nucleotide sequence, where the self-cleaving peptide encoding sequence is positioned between the first nucleic acid sequence and the second nucleic acid sequence.

As used herein, the term “self-cleaving peptide” refers to an 18-22 amino-acid long viral oligopeptide sequence that mediates ribosome skipping during translation in eukaryotic cells (Liu et al., “Systemic Comparison of 2A peptides for Cloning Multi-Genes in a Polycistronic Vector,” Scientific Reports 7: Article Number 2193 (2017), which is hereby incorporated by reference in its entirety). A non-limiting example of such a self-cleaving peptide is Peptide 2A, which is a short protein sequence first discovered in picornaviruses. Peptide 2A functions by making ribosomes skip the synthesis of a peptide bond at the C-terminus of a 2A element, resulting in a separation between the end of the 2A sequence and the peptide downstream thereof. This “cleavage” occurs between the glycine and proline residues at the C-terminus. Thus, successful ribosome skipping and recommencement of translation results in individual “cleaved” proteins where the protein upstream of the 2A element is attached to the complete 2A peptide except for the C-terminal proline and the protein downstream of the 2A element is attached to one proline at the N-terminus (Liu et al., “Systemic Comparison of 2A peptides for Cloning Multi-Genes in a Polycistronic Vector,” Scientific Reports 7: Article Number 2193 (2017), which is hereby incorporated by reference in its entirety).

Exemplary self-cleaving peptides that can be incorporated in the recombinant genetic construct include, without limitation, porcine teschovirus-1 2A (P2A), Foot and mouth disease virus 2A (F2A), Thosea asigna virus 2A (T2A), equine rhinitis A virus 2A (E2A), cytoplasmic polyhedrosis virus (BmCPV 2A), and flacherie virus (BmIFV 2A). Exemplary nucleotide sequences encoding self-cleaving peptides suitable for inclusion in the recombinant genetic construct described herein are provided in Table 4 below.

TABLE 4
Suitable Self-Cleaving Peptide Encoding Nucleotide Sequences

| Self-Cleaving | | SEQ |
|---|---|---|
| Peptide | Nucleotide Sequence* | ID NO. |
| Porcine | GGAAGCGGAG CTACTAACTT CAGCCTGCTG | 5 |
| teschovirus-1 2A | AAGCAGGCTG GAGACGTGGA GGAGAACCCT GGACCT | |
| (P2A) | | |
| Porcine | GGTTCCGGAG CCACGAACTT CTCTCTGTTA | 6 |
| teschovirus-1 2A | AAGCAAGCAG GAGACGTGGA AGAAAACCCC GGTCCC | |
| (P2A), codon | | |
| optimized | | |
| Foot and mouth | GGAAGCGGAG TGAAACAGAC TTTGAATTTT | 7 |
| disease virus 2A | GACCTTCTCA AGTTGGCGGG AGACGTGGAG | |
| (F2A) | TCCAACCCTG GACCT | |
| Thosea asigna virus | GAGGGCAGAG GAAGTCTTCT AACATGCGGT | 8 |
| 2A (T2A) | GACGTGGAGG AGAATCCCGG CCCT | |
| Equine rhinitis A | GGAAGCGGAC AGTGTACTAA TTATGCTCTC | 9 |
| virus 2A (E2A) | TTGAAATTGG CTGGAGATGT TGAGAGCAAC CCTGGACCT | |
| Cytoplasmic | GACGTTTTTC GCTCTAATTA TGACCTACTA | 10 |
| polyhedrosis virus | AAGTTGTGCG GTGATATCGA GTCTAATCCT GGACCT | |
| (BmCPV 2A) | | |
| Flacherie virus | ACTCTGACGA GGGCGAAGAT TGAGGATGAA | 11 |
| (BmIFV 2A) | TTGATTCGTG CAGGAATTGA ATCAAATCCT GGACCT | |

*See Wang et al., “2A Self-Cleaving Peptide-Based Multi-Gene Expression System in the Silkworm Bombyx mori,” Sci. Rep. 5:16273 (2015) and U.S. Pat. Application Publication No. 2018/0369280 to Schmitt et al., which are hereby incorporated by reference in their entirety.

In some embodiments of the methods and recombinant genetic constructs according to the present disclosure, the recombinant genetic construct encodes a posttranscriptional regulatory element sequence located 3′ to the nucleic acid sequence encoding the therapeutic molecule, the post-transcriptional modulator of gene expression, the phenoconversion-promoting molecule, the gene-editing molecule, and/or the epigenetic editing molecule. The posttranscriptional regulatory element may be selected from the group consisting of Woodchuck Hepatitis Virus (WHIP) Posttranscriptional Regulatory Element (WPRE), Hepatitis B virus Posttranscriptional Regulatory Element (TIPRE), human cytomegalovirus (hCMV) immediate/early (IE) intron A Posttranscriptional Regulatory Element, and any variants thereof.

In some embodiments, the posttranscriptional regulatory element is a Woodchuck Hepatitis Virus (WHIP) Posttranscriptional Regulatory Element (WPRE) having the sequence of substantially identical to the nucleic acid sequence identified by the Genflank Accession No. MQ250739.1 (which is hereby incorporated by reference in their entirety), or a portion thereof.

In some embodiments, the posttranscriptional regulatory element is a Hepatitis B virus Posttranscriptional Regulatory Element (HPRE) having the nucleic acid sequence of GenBank Accession No. GU253314.1 (which is hereby incorporated by reference in its entirety), or a portion thereof.

In some embodiments, the posttranscriptional regulatory element is a Hepatitis B virus Posttranscriptional Regulatory Element (HPRE) having the nucleic acid sequence of GenBank Accession No. GU253314.1 (which is hereby incorporated by reference in its entirety), or a portion thereof.

### Expression Vectors

Another aspect of the present disclosure is directed to an expression vector comprising the recombinant genetic construct according to the present disclosure. Such vectors include, without limitation, viral vectors, plasmid vectors, and bacterial vectors.

The term “vector” refers to a polynucleotide construct, typically a plasmid or a virus, used to transmit genetic material to a host cell. A vector as used herein can be composed of either DNA or RNA. In some embodiments, a vector is composed of DNA. An “expression vector” is a vector that is capable of directing the expression of sequence encoded by one or more nucleic acid sequences carried by the vector when it is present in the appropriate environment. Vectors may be capable of autonomous replication. Typically, an expression vector comprises a transcription promoter, a nucleic acid sequence encoding a therapeutic molecule, a post-transcriptional modulator of gene expression, a phenoconversion-promoting molecule, a gene-editing molecule, and/or an epigenetic editing molecule according to the present disclosure, and a transcription terminator. Expression of the therapeutic molecule, the post-transcriptional modulator of gene expression, the phenoconversion-promoting molecule, the gene-editing molecule, and/or the epigenetic editing molecule is usually placed under the control of a promoter, and a therapeutic molecule, a post-transcriptional modulator of gene expression, a phenoconversion-promoting molecule, a gene-editing molecule, and/or an epigenetic editing molecule is said to be “operably linked to” the promoter.

Suitable viral vectors include, without limitation, vaccina vectors, lentiviral vectors (integration competent or integration-defective lentiviral vectors), adenoviral vectors, adeno-associated viral vectors, vaccinia vectors, or any other vector suitable for introduction of the recombinant genetic construct described herein into a cell by any means to facilitate the gene/cell selective expression of the recombinant construct. In some embodiments of the methods and recombinant genetic constructs according to the present disclosure, the vector is a viral vector selected from the group consisting of a lentiviral vector, an adenoviral vector, an adeno-associated viral vector, and a vaccinia vector.

In some embodiments of the methods and recombinant genetic constructs according to the present disclosure, the vector is a lentiviral vector (see, e.g., U.S. Pat. No. 748,529 to Fang et al.; Ura et al., “Developments in Viral Vector-Based Vaccines,” Vaccines 2: 624-641 (2014); and Hu et al., “Immunization Delivered by Lentiviral Vectors for Cancer and Infection Diseases,” Immunol. Rev. 239: 45-61 (2011), which are hereby incorporated by reference in their entirety).

In some embodiments of the methods and recombinant genetic constructs according to the present disclosure, the vector is a retroviral vector (see, e.g., U.S. Pat. No. 748,529 to Fang et al., and Ura et al., “Developments in Viral Vector-Based Vaccines,” Vaccines 2: 624-641 (2014), which are hereby incorporated by reference in their entirety), a vaccinia virus, a replication deficient adenovirus vector, and a gutless adenovirus vector (see, e.g., U.S. Pat. No. 5,872,005, which is incorporated herein by reference in its entirety).

In some embodiments of the methods and recombinant genetic constructs according to the present disclosure, the vector is an adeno-associated virus (AAV) vector (see, e.g., Krause et al., “Delivery of Antigens by Viral Vectors for Vaccination,” Ther. Deliv. 2(1): 51-70 (2011); Ura et al., “Developments in Viral Vector-Based Vaccines,” Vaccines 2: 624-641 (2014); Buning et al, “Recent Developments in Adeno-associated Virus Vector Technology,” J. Gene Med. 10: 717-733 (2008), each of which is incorporated herein by reference in its entirety).

In some embodiments of the methods and recombinant genetic constructs according to the present disclosure, the vector is an adenoviral-associated viral (AAV) vector. A number of therapeutic AAV vectors suitable for delivery of the polynucleotides encoding antibodies described herein to the central nervous system are known in the art. See e.g., Deverman et al., “Gene Therapy for Neurological Disorders: Progress and Prospects,” Nature Rev. 17: 641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism. AAV vectors known to have tropism for the CNS include, AAV1, AAV2, AAV4, AAV5, AAV8 and AAV9 in their native form or engineered for enhanced tropism. In one embodiment, the AAV vector is an AAV2 vector. In another embodiment, the AAV vector is an AAV5 vector (Vitale et al., “Anti-tau Conformational scFv MC1 Antibody Efficiently Reduces Pathological Tau Species in Adult JNPL3 Mice,” Acta Neuropathol. Commun. 6: 82 (2018), which is hereby incorporate by reference in its entirety). In another embodiment, the AAV vector is an AAV9 vector (Haiyan et al., “Targeting Root Cause by Systemic scAAV9-hIDS Gene Delivery: Functional Correction and Reversal of Severe MPSII in Mice,” Mol. Ther. Methods Clin. Dev. 10: 327-340 (2018), which is hereby incorporated by reference in its entirety). In another embodiment, the AAV vector is an AAVrh10 vector (Liu et al., “Vectored Intracerebral Immunizations with the Anti-Tau Monoclonal Antibody PHF1 Markedly Reduces Tau Pathology in Mutant Transgenic Mice,” J. Neurosci. 36(49): 12425-35 (2016), which is hereby incorporated by reference in its entirety).

In some embodiments of the methods and recombinant genetic constructs according to the present disclosure, the AAV vector is a hybrid vector comprising the genome of one serotype, e.g., AAV2, and the capsid protein of another serotype, e.g., AAV1 or AAV3-9 to control tropism. See e.g., Broekman et al., “Adeno-Associated Virus Vectors Serotyped with AAV8 Capsid are More Efficient than AAV-1 or -2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector (Ising et al., “AAV-mediated Expression of Anti-Tau ScFv Decreases Tau Accumulation in a Mouse Model of Tauopathy,” J. Exp. Med. 214(5): 1227 (2017), which is hereby incorporated by reference in its entirety). In another embodiment the AAV vector is an AAV2/9 hybrid vector (Simon et al., “A Rapid Gene Delivery-Based Mouse Model for Early-Stage Alzheimer Disease-Type Tauopathy,” J. Neuropath. Exp. Neurol. 72(11): 1062-71 (2013), which is hereby incorporated by reference in its entirety).

In some embodiments of the methods and recombinant genetic constructs according to the present disclosure, the AAV vector is one that has been engineered or selected for its enhanced CNS transduction after intraparenchymal administration, e.g., AAV-DJ (Grimm et al., “In Vitro and In Vivo Gene Therapy Vector Evolution via Multispecies Interbreeding and Retargeting of Adeno-Associated Viruses,” J. Viol. 82: 5887-5911 (2008), which is hereby incorporated by reference in its entirety); increased transduction of neural stem and progenitor cells, e.g., SCH9 and AAV4.18 (Murlidharan et al., “Unique Glycan Signatures Regulate Adeno-Associated Virus Tropism in the Developing Brain,” J. Virol. 89: 3976-3987 (2015) and Ojala et al., “In Vivo Selection of a Computationally Designed SCHEMA AAV Library Yields a Novel Variant for Infection of Adult Neural Stem Cells in the SVZ,” Mol. Ther. 26: 304-319 (2018), which are hereby incorporated by reference in their entirety); enhanced retrograde transduction, e.g., rAAV2-retro (Muller et al., “Random Peptide Libraries Displayed on Adeno-Associated Virus to Select for Targeted Gene Therapy Vectors,” Nat. Biotechnol. 21: 1040-1046 (2003), which is hereby incorporated by reference in its entirety); selective transduction into brain endothelial cells, e.g., AAV-BRI (Körbelin et al., “A Brain Microvasculature Endothelial Cell-Specific Viral Vector with the Potential to Treat Neurovascular and Neurological Diseases,” EMBO Mol. Med. 8(6): 609-625 (2016), which is hereby incorporated by reference in its entirety); or enhanced transduction of the adult CNS after IV administration, e.g., AAV-PHP.B and AAVPHP.eB (Deverman et al., “Cre-Dependent Selection Yields AAV Variants for Widespread Gene Transfer to the Adult Brain,” Nat. Biotechnol. 34(2): 204-209 (2016) and Chan et al., “Engineered AAVs for Efficient Noninvasive Gene Delivery to the Central and Peripheral Nervous Systems,” Nat. Neurosci. 20(8): 1172-1179 (2017), which are hereby incorporated by reference in their entirety.

Methods for generating and isolating viral expression vectors suitable for use as vectors are known in the art (see, e.g., Bulcha et al., “Viral Vector Platforms within the Gene Therapy Landscape,” Nature 6(1): 53 (2021); Bouard et al., “Viral Vectors: From Virology to Transgene Expression,” Br. J. Pharmacol. 157(2): 153-165 (2009); Grieger & Samulski, “Adeno-associated Virus as a Gene Therapy Vector: Vector Development, Production and Clinical Applications,” Adv. Biochem. Engin Biotechnol. 99: 119-145 (2005); Buning et al, “Recent Developments in Adeno-associated Virus Vector Technology,” J. Gene Med. 10(7): 717-733 (2008), each of which is incorporated herein by reference in its entirety).

### Pharmaceutical Compositions

A further aspect of the present disclosure is directed to a pharmaceutical composition comprising the recombinant genetic construct or the expression vector according to the present disclosure and a pharmaceutically acceptable carrier.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency or listed in the U.S. Pharmacopeia or other generally recognized pharmacopeia for use in mammals, and more particularly in humans.

A pharmaceutically acceptable “carrier” refers to a diluent, adjuvant, excipient, or vehicle with which the compound is administered. Such pharmaceutically acceptable carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutically acceptable carriers are described in “Remington's Pharmaceutical Sciences” by E. W. Martin.

Examples of pharmaceutically acceptable carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

In some embodiments of the methods and pharmaceutical compositions according to the present disclosure, compositions are formulated for parenteral administration, e.g., intraventricular, intracallosal, or intraparenchymal administration. In some embodiments, the composition is reconstituted from a lyophilized preparation prior to administration.

For parenteral administration, pharmaceutical compositions of the present disclosure are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the disclosure can be administered in the form of a depot injection or implant preparation which can be formulated in such a manner as to permit a sustained release of the active ingredient.

### Preparations of Cells

Another aspect of the present disclosure relates to a preparation of cells comprising glial progenitor cells, where cells of the preparation comprise a recombinant genetic construct or expression vector according to the present disclosure or an expression vector according to the present disclosure.

As used herein, the term “glial cells” refers to a population of non-neuronal cells that provide support and nutrition, maintain homeostasis, either form myelin or promote myelination, and participate in signal transmission in the nervous system. The term “glial progenitor cells” refers to cells having the potential to differentiate into cells of the glial lineage such as oligodendrocytes and astrocytes (French-Constant and Raff, “Proliferating Bipotential Glial Progenitor Cells in Adult Rat Optic Nerve,” Nature 319: 499-502 (1986) and Raff et al., “A Glial Progenitor Cell that Develops in Vitro into an Astrocyte or an Oligodendrocyte Depending on Culture Medium,” Nature 303: 390-396 (1983), which are hereby incorporated by reference in their entirety).

The glial progenitor cells of the preparation may be astrocyte biased glial progenitor cells, oligodendrocyte-biased glial progenitor cells, unbiased glial progenitor cells, or a combination thereof. The glial progenitor cells of the preparation may express one or more markers of the glial cell lineage. For example, in one embodiment, the glial progenitor cells of the preparation may express A2B5⁺. In another embodiment, the glial progenitor cells of the preparation are positive for a PDGFαR marker. The PDGFαR marker is optionally a PDGFαR ectodomain, such as CD140a. PDGFαR and CD140a are markers of an oligodendrocyte-biased glial progenitor cells. In another embodiment, the glial progenitor cells of the preparation are CD44⁺. CD44 is a marker of an astrocyte-biased glial progenitor cell. In another embodiment, the glial progenitor cells of the preparation are positive for a CD9 marker. The CD9 marker is optionally a CD9 ectodomain. In one embodiment, the glial progenitor cells of the preparation are A2B5⁺, CD140a⁺, and/or CD44⁺. The aforementioned glial progenitor cell surface markers can be used to identify, separate, and/or enrich the preparation for glial progenitor cells prior to administration.

The glial progenitor cell preparation is optionally negative for a PSA-NCAM marker and/or other neuronal lineage markers, and/or negative for one or more inflammatory cell markers, e.g., negative for a CD11 marker, negative for a CD32 marker, and/or negative for a CD36 marker (which are markers for microglia). Optionally, the preparation of glial progenitor cells are negative for any combination or subset of these additional markers. Thus, for example, the preparation of glial progenitor cells is negative for any one, two, three, or four of these additional markers.

Glial progenitor cells of the preparation may be stably transduced with one or more of the recombinant genetic constructs described herein. In accordance with such embodiments, the glial progenitor cells of the preparation express at least one of the recombinant genetic constructs described herein. In some embodiments, the therapeutic molecule, the post-transcriptional modulator of gene expression, the phenoconversion-promoting molecule, the gene-editing molecule, and/or the epigenetic editing molecule encoded by each of the one or more recombinant genetic constructs is not endogenously expressed by the glial progenitor cells of the preparation; however, the therapeutic molecule, the post-transcriptional modulator of gene expression, the phenoconversion-promoting molecule, the gene-editing molecule, and/or the epigenetic editing molecule is expressed in a target cell-specific manner via the activation of the GPR17 promoter-inclusive regulatory element.

The preparation of glial progenitor cells may be a preparation of glial progenitor cells from any organism. In some embodiments, the preparation of glial progenitor cells is a preparation of mammalian cells, e.g., a preparation of rodent cells (i.e., mouse or rat cells), rabbit cells, guinea pig cells, feline cells, canine cells, porcine cells, equine cells, bovine cell, ovine cells, monkey cells, or human cells. In one embodiment, the preparation of glial progenitor cells is a preparation of human glial progenitor cells.

Glial progenitor cells can be obtained from embryonic, fetal, or adult brain tissue, embryonic stem cells, or induced pluripotential cells. Suitable methods for obtaining glial progenitor cells from embryonic stem cells or induced pluripotent stem cells are known in the art, see e.g., U.S. Pat. No. 10,450,546 to Goldman and Wang, which is hereby incorporated by reference in its entirety.

Alternatively, the glial progenitor cells are isolated from ventricular and subventricular zones of the brain or from the subcortical white matter. Glial progenitor cells can be extracted from brain tissue containing a mixed population of cells directly by using the promoter specific separation technique, as described in U.S. Patent Application Publication Nos. 20040029269 and 20030223972 to Goldman, which are hereby incorporated by reference in their entirety. This method involves selecting a promoter which functions specifically in glial progenitor cells, and introducing a nucleic acid encoding a marker protein under the control of said promoter into the mixed population cells. The mixed population of cells is allowed to express the marker protein and the cells expressing the marker protein are separated from the population of cells, with the separated cells being the glial progenitor cells.

In some embodiments of the methods and preparation of cells according to the present disclosure, the preparation of glial progenitor cells is a preparation of bi-potential glial progenitor cells.

In some embodiments of the methods and preparation of cells according to the present disclosure, the preparation of glial progenitor cells is biased to producing oligodendrocytes. In accordance with such embodiments, the preparation of glial progenitor cells may be oligodendrocyte progenitor cells.

Alternatively, the glial progenitor cells are biased to producing astrocytes.

In some embodiments of the methods and preparation of cells according to the present disclosure, cells of the preparation are transduced with a recombinant genetic construct encoding a therapeutic molecule, a reporter molecule, a post-transcriptional modulator of gene expression, a phenoconversion-promoting molecule, an gene-editing molecule, and an epigenetic editing molecule. Suitable therapeutic molecules, reporter molecules, post-transcriptional modulators of gene expression, phenoconversion-promoting molecules, gene-editing molecules, and epigenetic editing molecules are described in more detail supra.

In accordance with this aspect of the disclosure, the recombinant genetic construct may be integrated into the chromosome of the one or more cells in the preparation. The term “integrated,” when used in the context of the recombinant genetic construct of the present disclosure means that the recombinant genetic construct is inserted into the genome or the genomic sequence of the one or more cells in the preparation. When integrated, the integrated recombinant genetic construct is replicated and passed along to daughter cells of a dividing cell in the same manner as the original genome of the cell.

Another aspect of the present disclosure relates to a method of delivering a nucleic acid construct encoding a protein of interest to glial progenitor cells. This method involves providing a recombinant genetic construct comprising: (i) a G-protein coupled receptor (GPR17) promoter-inclusive regulatory element sequence having a 5′ and a 3′ end and (ii) a nucleic acid sequence encoding the protein of interest, where the nucleic acid sequence encoding the protein of interest is heterologous to the GPR17 promoter-inclusive regulatory element and where the nucleic acid sequence encoding the protein of interest is positioned 3′ to the GPR17 promoter-inclusive regulatory element sequence. The method further involves transfecting or transducing the glial progenitor cells with the recombinant genetic construct.

Suitable GPR17 promoter-inclusive regulatory elements are described in more detail supra. In some embodiments, the GPR17 promoter-inclusive regulatory element comprises the sequence of SEQ ID NO: 2. In some embodiments, the GPR17 promoter-inclusive regulatory element comprises the sequence of SEQ ID NO: 1.

Suitable methods for transfecting and transducing cells are well known in the art. In some embodiments, said transfecting or transducing is carried out ex vivo. In some embodiments, said transfecting or transducing is carried out in vivo.

The method may be carried out by transfection with a virus selected from the group consisting of a lentivirus, an adenovirus, an adeno-associated virus, and a vaccinia virus.

In some embodiments, the method is carried out to treat a mammalian subject for a condition mediated by glial progenitor cells. Suitable conditions mediated by glial progenitor cells are described in detail supra.

Another aspect of the present disclosure relates to a method of delivering a nucleic acid construct encoding a protein of interest to astrocytes and glial progenitor cells. This method involves providing a recombinant genetic construct comprising: (i) a G-protein coupled receptor (GPR17) promoter-inclusive regulatory element sequence having a 5′ and a 3′ end; (ii) a glial fibrillary acidic protein (GFAP) promoter-inclusive regulatory element; and (ii) a nucleic acid sequence encoding the protein of interest, where the nucleic acid sequence encoding the protein of interest is heterologous to the GPR17 promoter-inclusive regulatory element as well as to the GFAP promoter-inclusive regulatory element and where the GFAP promoter-inclusive regulatory element is positioned within the recombinant genetic construct between the GPR17 promoter-inclusive regulatory element sequence and the nucleic acid sequence encoding the protein of interest. This method further involves transfecting or transducing a population of glial progenitor cells with the recombinant genetic construct, where (a) prior to differentiation of the transfected or transduced glial progenitor cells, the nucleic acid sequence encoding said protein of interest is expressed under control of the GPR17 promoter-inclusive regulatory element and (b) after differentiation of the transfected or transduced glial progenitor cells to astrocytes, said protein of interest is expressed under control of the GFAP promoter-inclusive regulatory element.

Suitable GPR17 promoter-inclusive regulatory elements are described in more detail supra. In some embodiments, the GPR17 promoter-inclusive regulatory element comprises the sequence of SEQ ID NO: 2. In some embodiments, the GPR17 promoter-inclusive regulatory element comprises the sequence of SEQ ID NO: 1. In some embodiments, the GFAP promoter-inclusive regulatory element has the sequence of SEQ ID NO: 3.

Suitable methods for transfecting and transducing cells are well known in the art. In some embodiments, said transfecting or transducing is carried out ex vivo. In some embodiments, said transfecting or transducing is carried out in vivo.

The method may be carried out by transfection with a virus selected from the group consisting of a lentivirus, an adenovirus, an adeno-associated virus, and a vaccinia virus.

In some embodiments, the method is carried out to treat a mammalian subject for a condition mediated by astrocytes. Suitable conditions mediated by astrocytes are described in detail supra. For example, the condition mediated by astroctytes may be selected from the group consisting of Huntington's disease (HD), Parkinson's disease (PD), Alzheimer's disease (AD), and amyotrophic lateral sclerosis (ALS).

### EXAMPLES

The examples below are intended to exemplify the practice of embodiments of the disclosure but are by no means intended to limit the scope thereof.

### Example 1—Design of Cell-Specific Viral Vector for Glial Progenitor Cells

Lentiviral expression vectors LV-GFAP-EGFP-M124T, LV-GPR17(2.2)-EGFP-M124T, LV-GPR17(0.8)-EGFP-M124T, and LV-GPR17-GFAP-EGFP-M124T were designed to express a protein of interest in an astrocyte and/or glial progenitor cell-specific manner.

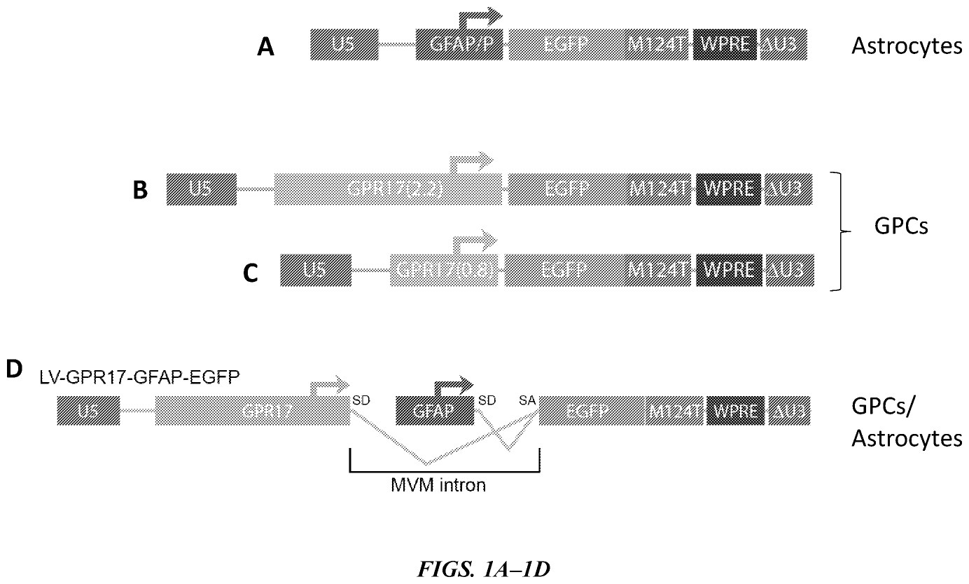

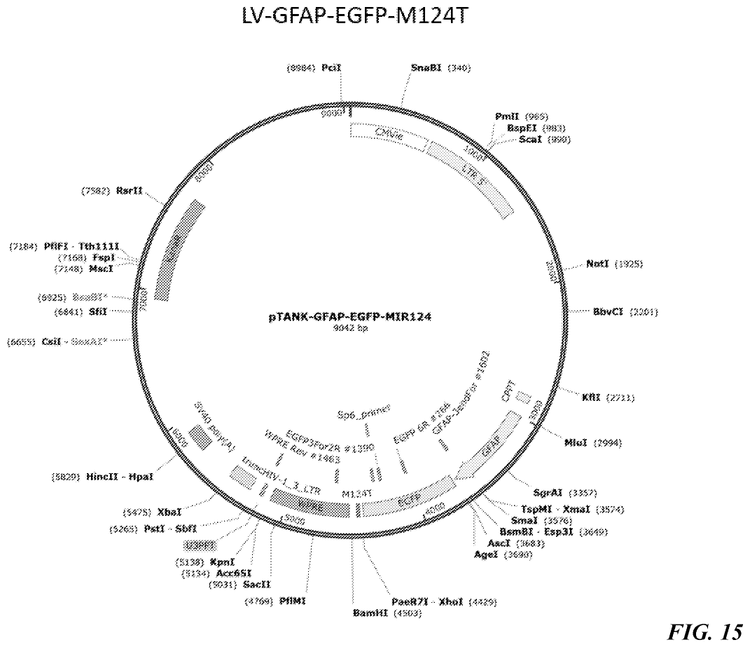

LV-GFAP-EGFP-M124T, comprising, in the 5′ to 3′ direction, a glial fibrillary acidic protein (GFAP; gfaABC₁D) promoter-inclusive regulatory element (Lee et al., “GFAP Promoter Elements Required for Region-Specific and Astrocyte-Specific Expression,” Glia 56(5):481-93 (2008), which is hereby incorporated by reference in its entirety); a nucleic acid sequence encoding enhanced green fluorescent protein (EGFP); MIR124 target that allows silencing of the vector in neurons; and post translational regulatory element WPRE for transcript stability in the cells was designed for targeting astrocytes (FIG. 1A; FIG. 15).

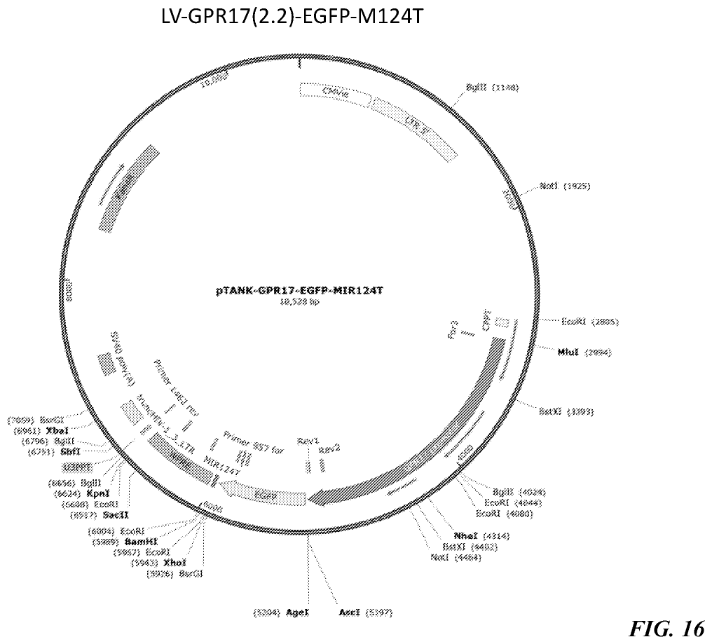

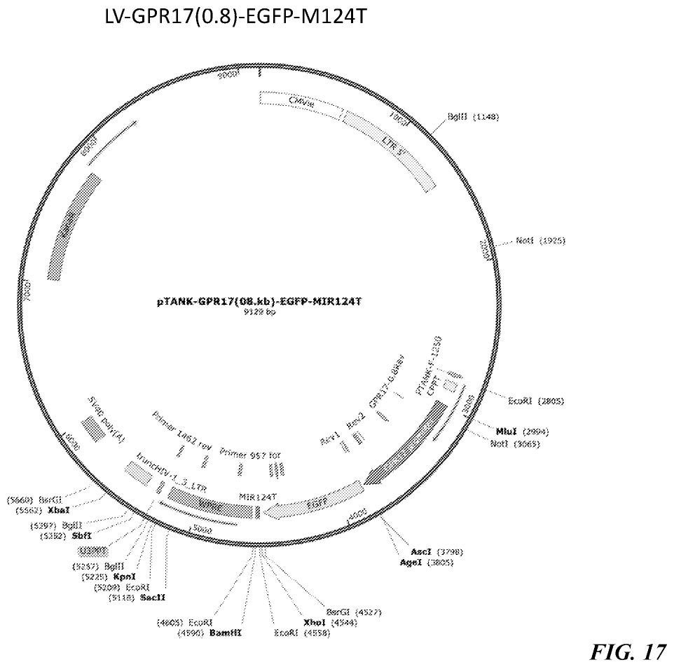

LV-GPR17(2.2)-EGFP-M124T and LV-GPR17(0.8)-EGFP-M124T comprising, in the 5′ to 3′ direction, a G-protein coupled receptor (GPR17) promoter-inclusive regulatory element having the sequence of SEQ ID NO: 1 (2.2 kb; FIG. 1B, FIG. 16) or SEQ ID NO: 2 (0.8 kb; FIG. 1C, FIG. 17); a nucleic acid sequence encoding enhanced green fluorescent protein (EGFP); MIR124 target that allows silencing of the vector in neurons; and post translational regulatory element WPRE for transcript stability in the cells were designed for targeting glial progenitor cells (FIG. 1, FIG. 16; FIG. 1C, FIG. 17).

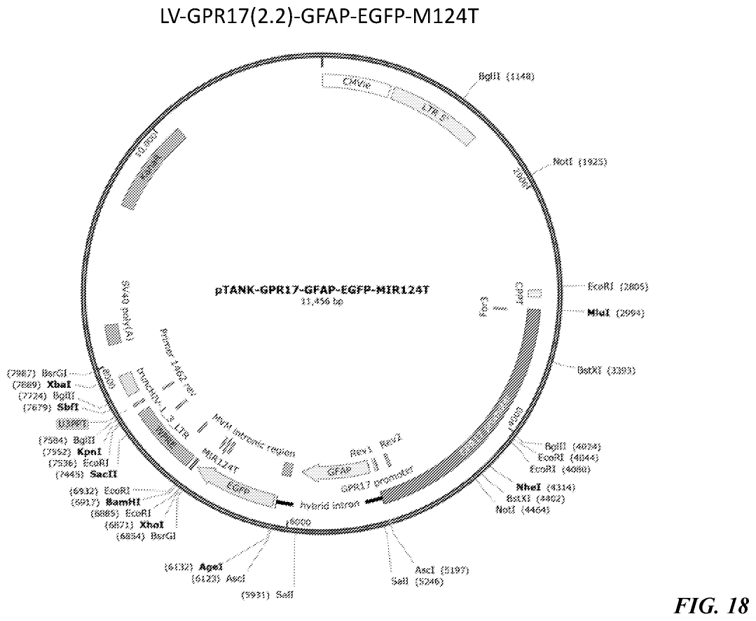

LV-GPR17(2.2)-GFAP-EGFP-M124T comprising, in the 5′ to 3′ direction, a G-protein coupled receptor (GPR17) promoter-inclusive regulatory element having the sequence of SEQ ID NO: 1 (2.2 kb); a glial fibrillary acidic protein (GFAP; gfaABC₁D) promoter-inclusive regulatory element; a nucleic acid sequence encoding enhanced green fluorescent protein (EGFP); MIR124 target that allows silencing of the vector in neurons; and post translational regulatory element WPRE for transcript stability in the cells were designed for targeting both astrocytes and glial progenitor cells (FIG. 1D; FIG. 18). As shown in FIG. 1D, the GFAP promoter-inclusive regulatory element was inserted in intron (Small intron of Minute virus mouse) to allow for alternative splicing of the transcripts emanating from the two promoters (Haut et al., “Intron Definition is Required for Excision of the Minute Virus of Mice Small Intron and Definition of the Upstream Exon,” J. Virol. 72(3):1834-1843 (1998), which is hereby incorporated by reference in its entirety).

All four viruses were amplified and viral particles were pseudo-typed with VSV-G viral envelope.

### Example 2—LV-GFAP-EGFP-M124T Drives Expression of EGFP In Vivo

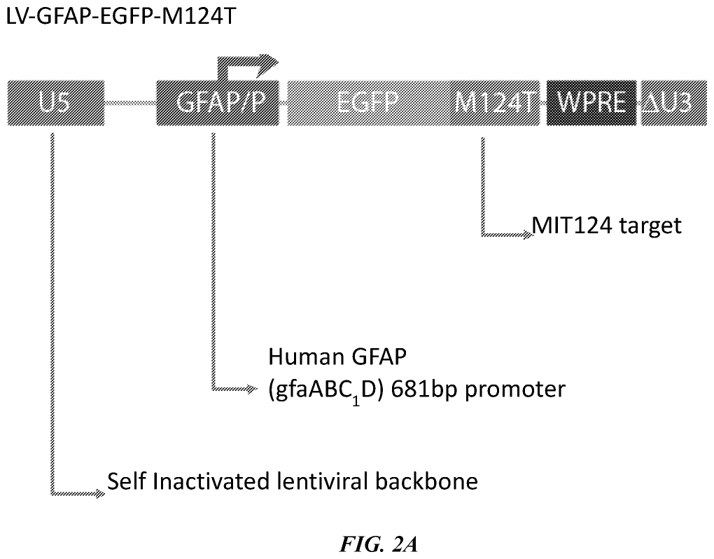

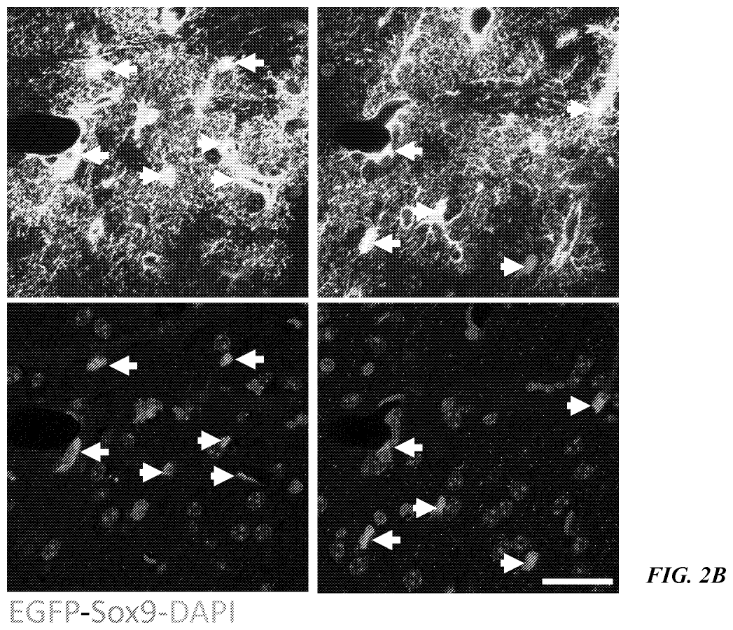

To determine whether LV-GFAP-EGFP-M124T (FIG. 2A) enables astrocyte expression in vivo, one microliter of viral suspension of LV-GFAP-EGFP-M124T were injected in the striata of 10 weeks old mice. The mice were sacrificed one week later, and brain tissue was processed for histology. Immuno-fluorescence staining with Sox9, a cell specific marker of astrocytes, showed that all EGFP is expressed exclusively in Sox9-expressing astrocytes (FIG. 2B).

### Example 3—LV-GPR17(2.2)-EGFP-M124T Drives Expression of EGFP In Vivo

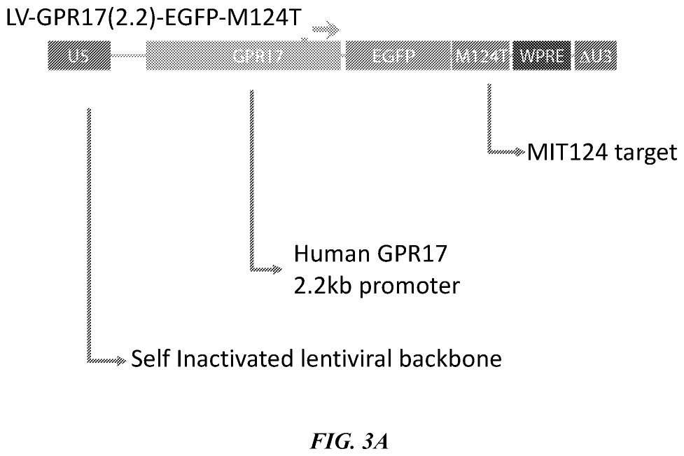

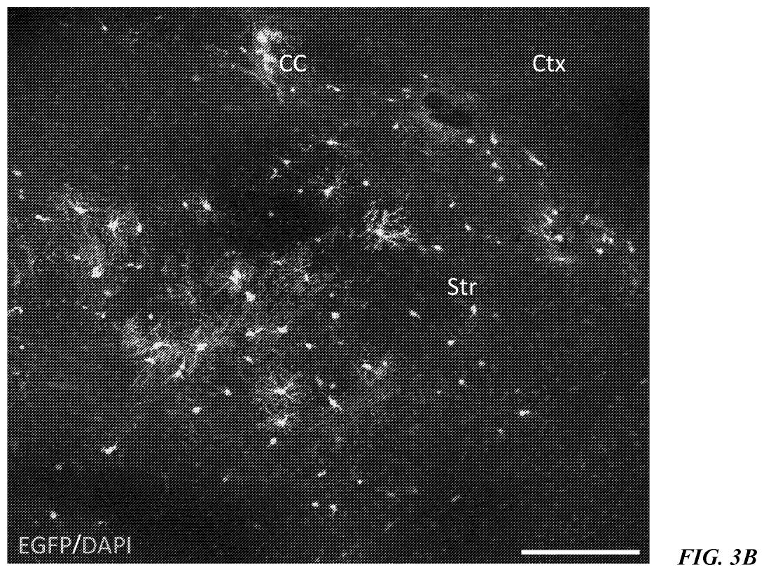

To determine whether LV-GPR17(2.2)-EGFP-M124T (FIG. 3A) enables glial progenitor cells expression in vivo, one microliter of viral suspension of LV-GPR17(2.2)-EGFP-M124T was injected in the striata of 10 weeks old mice. The mice were sacrificed one week later, and brain tissue was processed for histology. Fluorescence microscopy demonstrated that EGFP was expressed in the striatum and corpus callosum (FIG. 3B).

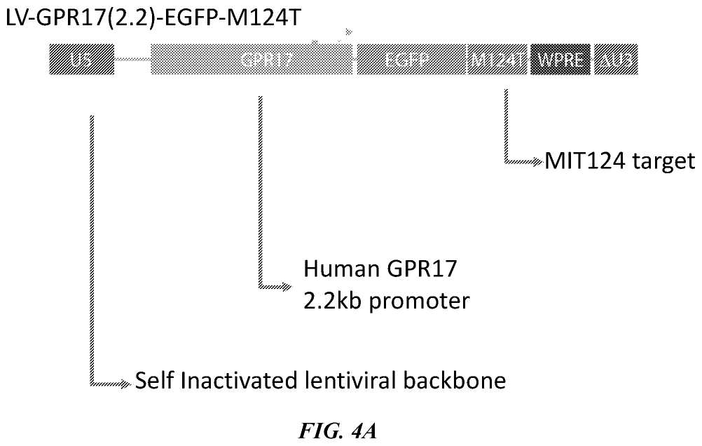

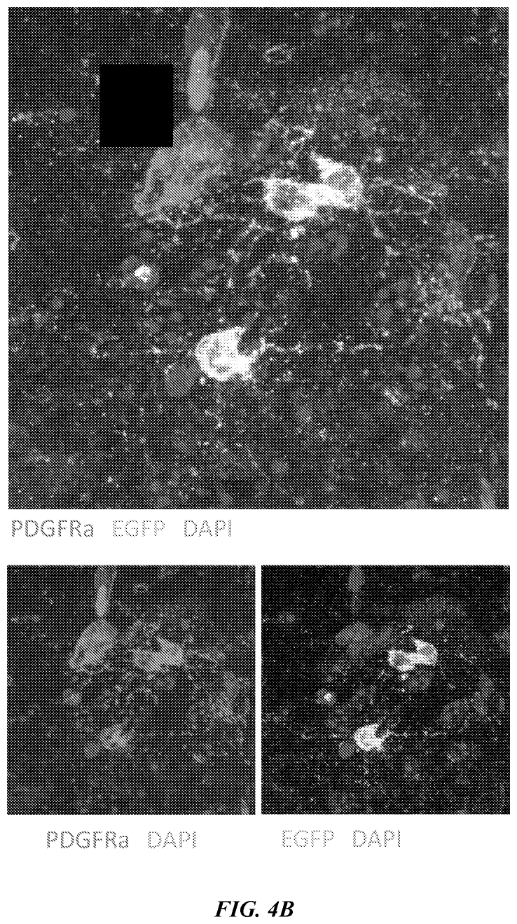

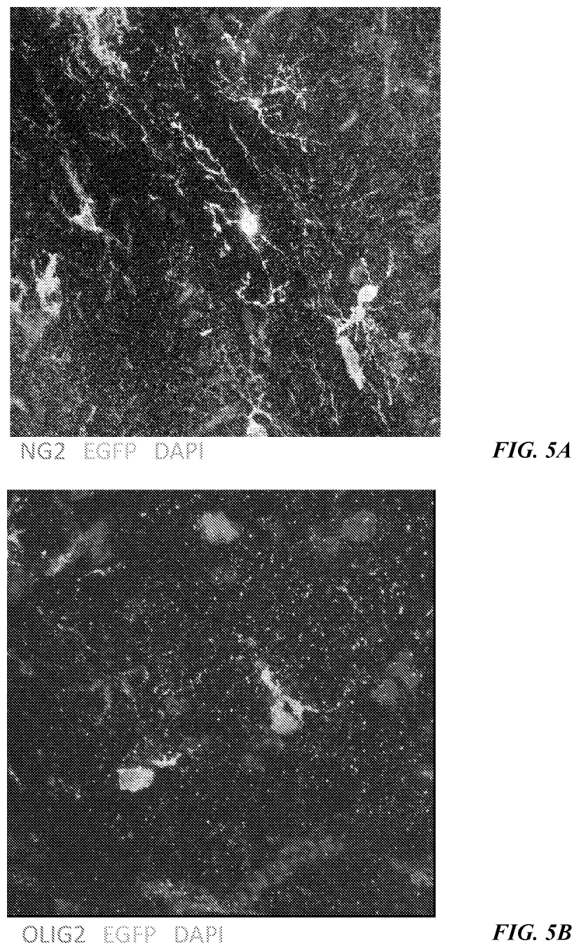

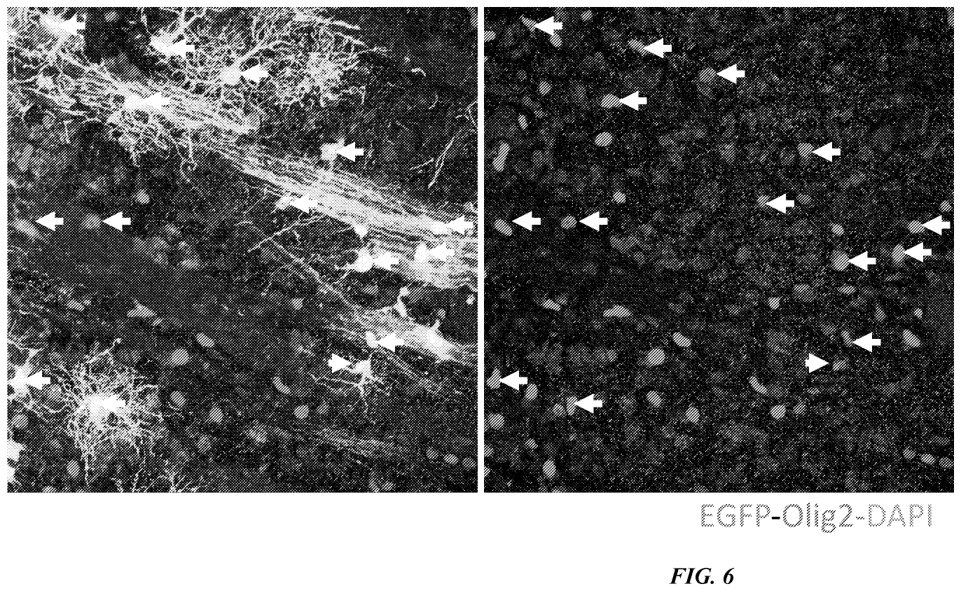

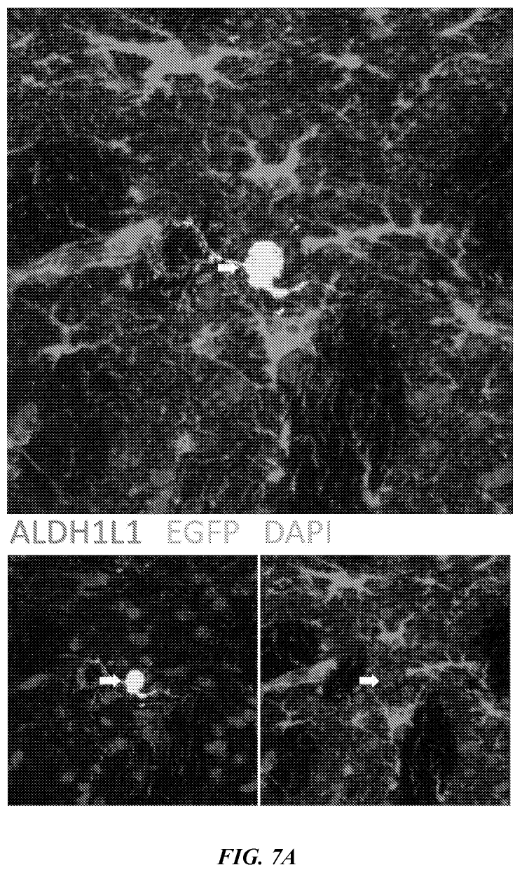

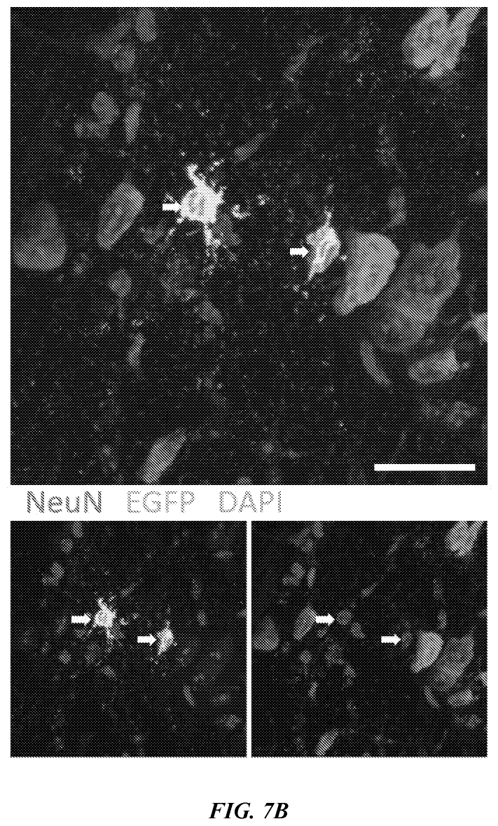

LV-GPR17(2.2)-EGFP-MT124T (FIG. 4A) expression is restricted to glial progenitor cells (GPCs)/oligodendrocyte progenitor cells (OPCs) in adult mouse brain as shown by colocalization of EGFP and PDGFRa immune-staining (FIG. 4B). GPR17 promoter-driven EGFP expression occurs in oligodendrocytes co-expressing NG2 (FIG. 5A) and Olig2 (FIG. 5B), two specific markers that define OPCs and oligodendroglia cells, respectively. Striatally-injected LV-GPR17(2.2)-EGFP-MT124T expression is restricted to striatal Olig2 expressing glial progenitor cells and young oligodendrocytes (FIG. 6). EGFP+/Olig2+ cells showed typical morphology of OPCs and young oligodendrocytes (FIG. 6). Of note, LV-GPR17(2.2)-EGFP-MT124T is not expressed in ALDH1L1⁺ astrocytes (FIG. 7A) or NeuN⁺ neurons (FIG. 7B) in the adult mouse brain.

### Example 4—LV-GPR17(0.8)-EGFP-M124T Drives Expression of EGFP In Vivo

LV-GPR17(2.2)-EGFP-M124T vector comprises a 2.2 kb fragment of the GPR17 promoter-inclusive regulatory element, which can only be used in viruses that allow large DNA insertion (e.g., Lentivirus or Adenovirus). The size limit is easily exceeded for viruses that only can express small transgenes (i.e., Adenoassociated virus, 4.5 Kb). Thus, whether a shorter fragment of GPR17 prompter-inclusive regulatory element, consisting of 0.8 kb immediately upstream of the TSS is sufficient to drive cell-specific expression of a reporter similar to that of the longer 2.2 kb promoter was next investigated.

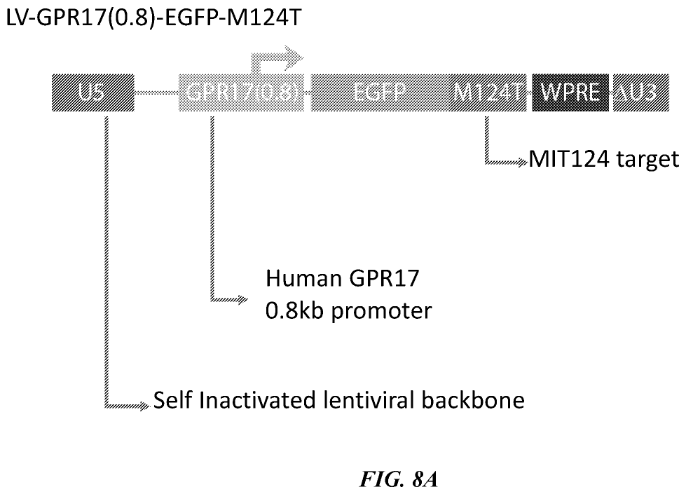

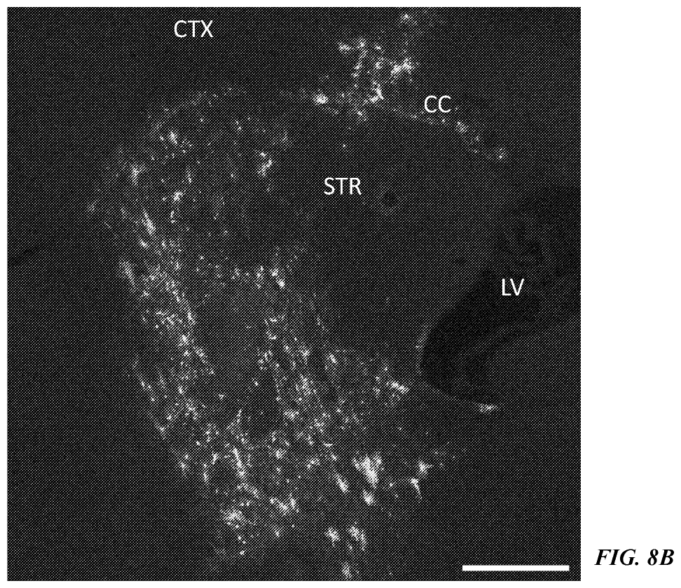

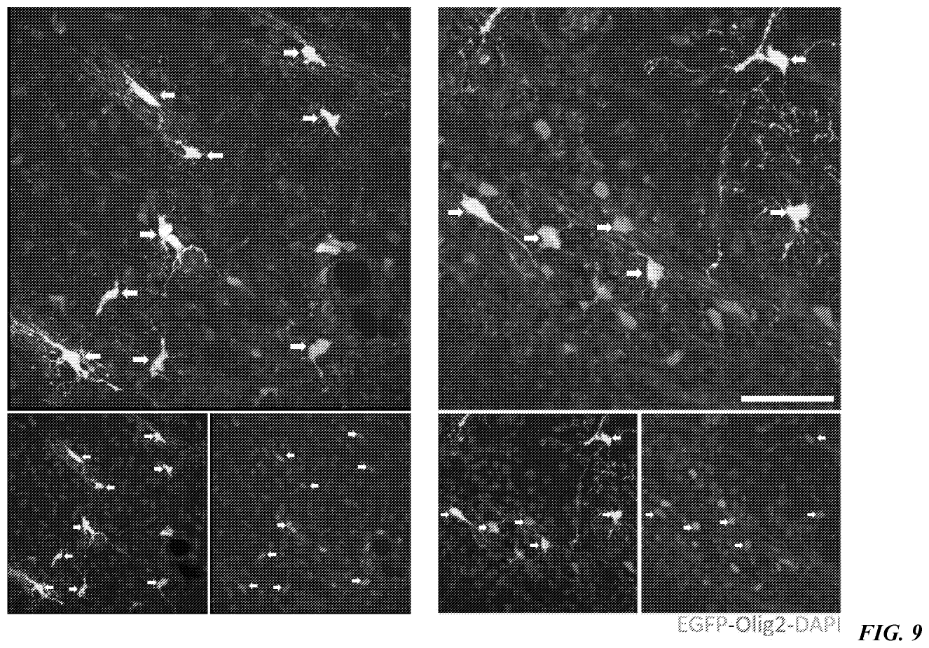

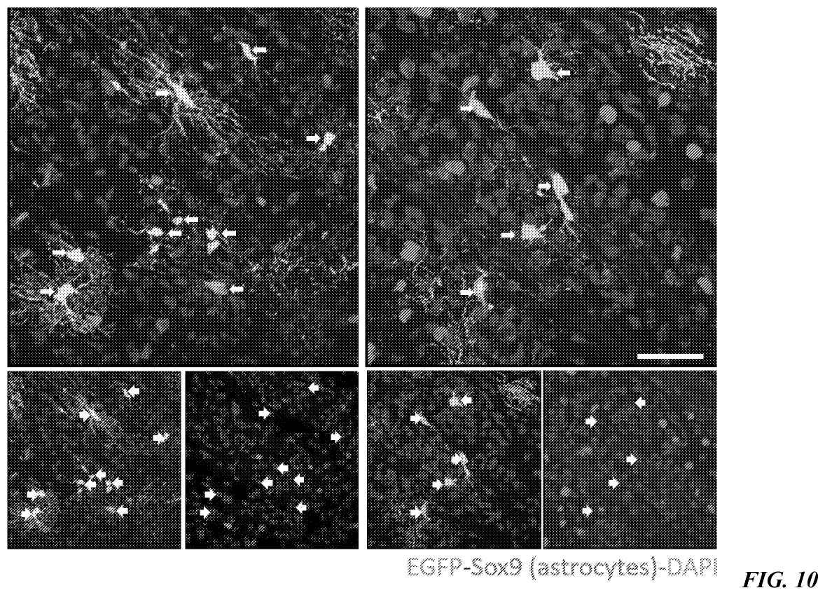

LV-GPR17(0.8)-EGFP-M124T (FIG. 8A) was injected in the striata of 10 weeks old mice. The mice were sacrificed one week later, and brain tissue was processed for histology. Fluorescence microscopy demonstrated that EGFP was expressed in the striatum (FIG. 8B); LV-GPR17(0.8)-EGFP-M124T maintains specific expression in Olig2⁺ glial progenitor cells in vivo (FIG. 9); and that EGFP is not expressed from LV-GPR17(0.8)-EGFP-M124T in Sox9⁺ astrocytes in the adult mouse striatum (FIG. 10).

### Example 5—LV-GPR17(2.2)-GFAP-EGFP-M124T Drives Expression of EGFP In Vivo

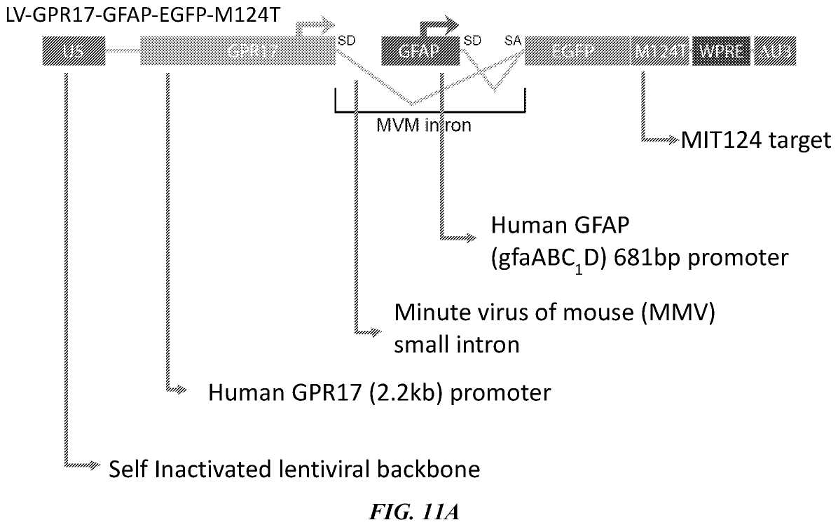

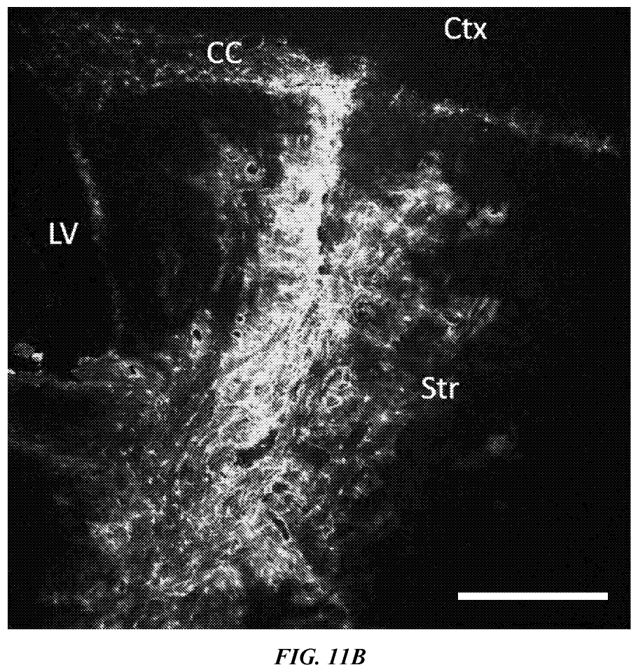

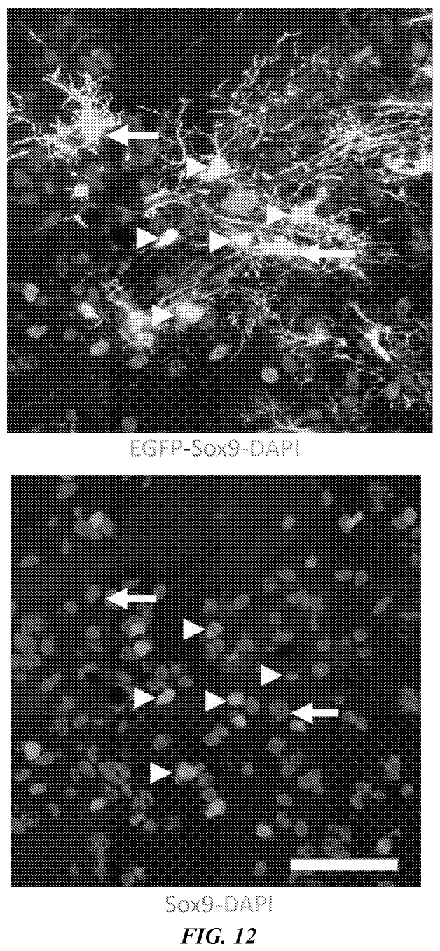

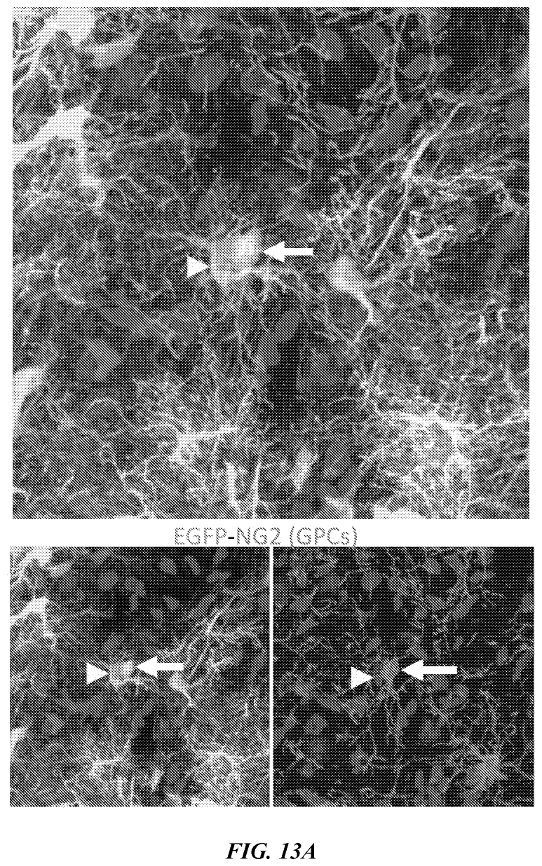

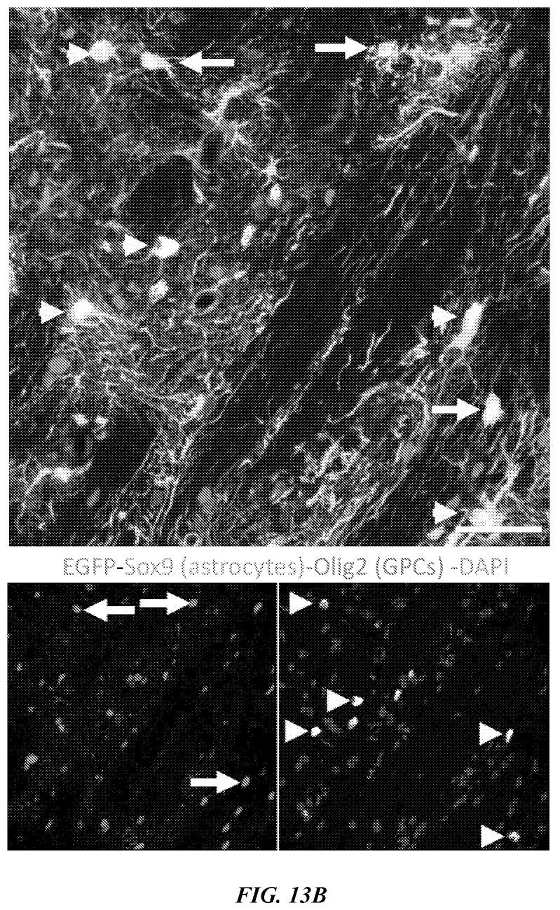

To investigate whether a lentiviral construct could enable glial progenitor cell and astrocyte-specific expression in vivo, a lentiviral vector in which GPR17 and GFAP promoter are assembled in tandem to drive the expression of the same reporter (i.e., EGFP) by alternate splicing of transcripts was designed (FIG. 11A). A viral suspension of LV-GPR17(2.2)-GFAP-EGFP-M124T was injected in the striata of 10 weeks old mice. The mice were sacrificed one week later, and brain tissue was processed for histology. Fluorescence microscopy demonstrated that EGFP was expressed in the striatum and corpus callosum (FIG. 11B); LV-GPR17(2.2)-EGFP-M124T is expressed in Sox9⁺ astrocytes, but not in Sox9⁻ cells in the adult mouse striatum (FIG. 12); that EGFP expression only partially colocalizes with NG2-expressing glial progenitor cells (FIG. 13A); and that EGFP⁺ cells comprise both Sox9⁺ astrocytes and Olig2⁺ oligodendroglial lineage cells (FIG. 13B).

### Example 6—Cell Type Specificity of Promoter-Based Lentiviral Targeting Vectors

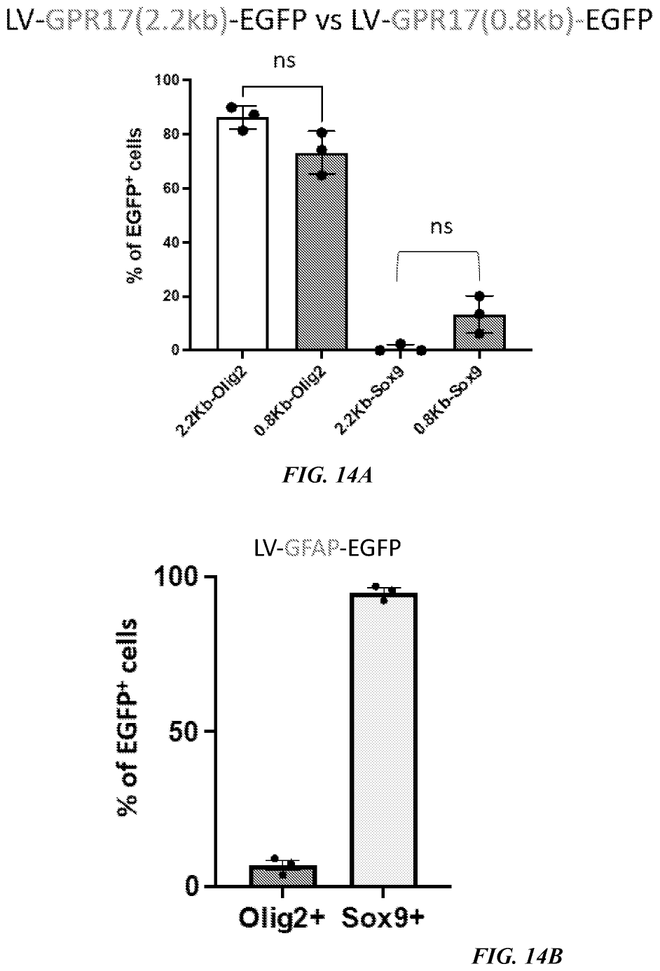

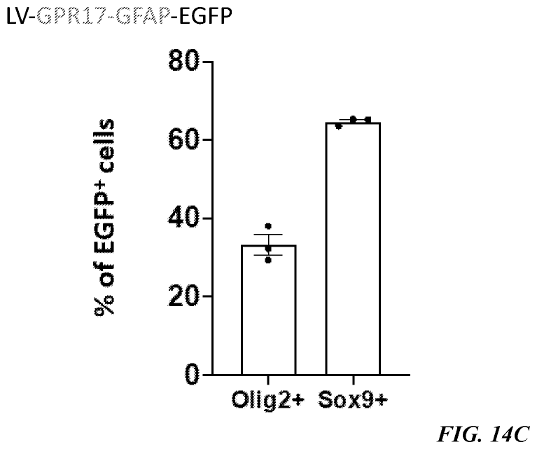

To investigate the cell type specificity of LV-GFAP-EGFP-M124T, LV-GPR17(2.2)-EGFP-M124T, LV-GPR17(0.8)-EGFP-M124T, and LV-GPR17-GFAP-EGFP-M124T vectors, the distribution of EGFP⁺ cells in the striata of mice injected with each construct was evaluated. LV-GPR17(2.2)-EGFP-M124T and LV-GPR17(0.8)-EGFP-M124T both similarly transduced Sox9⁺ astrocytes and Olig2⁺ oligodendroglial cells (FIG. 14A); LV-GFAP-EGFP-M124T drove expression predominantly in Sox9-expressing astrocytes (FIG. 14B); and the dual promoter lentivirus LV-GPR17(2.2)-GFAP-EGFP-M124T droves expression in both Sox9⁺ astrocytes and Olig2⁺ oligodendroglia lineage cells.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating a disease or disorder in a subject in need thereof, said method comprising:
providing a recombinant genetic construct comprising:
(i) a G-protein coupled receptor (GPR17) promoter-inclusive regulatory element sequence having a 5′ and a 3′ end and
(ii) a nucleic acid sequence encoding a molecule of interest, wherein said nucleic acid sequence is heterologous to the GPR17 promoter-inclusive regulatory element and wherein said nucleic acid sequence encoding the molecule of interest is positioned 3′ to the GPR17 promoter-inclusive regulatory element sequence; an expression vector comprising the recombinant genetic construct; a pharmaceutical composition comprising the recombinant genetic construct or the expression vector; or a preparation of cells comprising the recombinant genetic construct or the expression vector and
administering, to the subject in need, an effective amount of the recombinant genetic construct, the expression vector, the pharmaceutical composition, or the preparation of cells.

2. The method of claim 1, wherein the molecule of interest is selected from the group consisting of a therapeutic molecule, a post-transcriptional modulator of gene expression, a phenoconversion-promoting molecule, a gene-editing molecule, and an epigenetic editing molecule.

3. The method of claim 1, wherein the recombinant genetic construct, the expression vector, the pharmaceutical composition, or the preparation of cells is administered to one or more sites of the subject's brain, brain stem, spinal cord, or a combination thereof, or is administered intraventricularly, intracallosally, or intraparenchymally.

4. (canceled)

5. The method of claim 1, wherein the subject has a disease or disorder selected from the group consisting of a vascular disorder, a neuroimmune disorder, a neurodegenerative disorder, and a neuropsychiatric disease of neuronal loss, or
has a neurodegenerative disorder selected from the group consisting of Huntington's disease, frontotemporal dementia, Parkinson's disease, multisystem atrophy, and amyotrophic lateral sclerosis, or
has a neuropsychiatric disorder selected from the group consisting of schizophrenia, autism spectrum disorder, and bipolar disorder, or
has a human myelin disease, wherein the myelin disease is a leukodystrophy or a white matter disease.

6-8. (canceled)

9. A recombinant genetic construct comprising:
(i) a G-protein coupled receptor (GPR17) promoter-inclusive regulatory element sequence having a 5′ and a 3′ end and
(ii) a nucleic acid sequence encoding a molecule of interest, wherein said nucleic acid sequence encoding a molecule of interest is heterologous to the GPR17 promoter-inclusive regulatory element and wherein said nucleic acid sequence encoding the molecule of interest is positioned 3′ to the GPR17 promoter-inclusive regulatory element sequence.

10. The recombinant genetic construct of claim 9, wherein the molecule of interest is selected from the group consisting of a therapeutic molecule, a post-transcriptional modulator of gene expression, a phenoconversion-promoting molecule, a gene-editing molecule, and an epigenetic editing molecule.

11. The recombinant genetic construct of claim 9, wherein the GPR17 promoter-inclusive regulatory element has the sequence of SEQ ID NO: 2 or SEQ ID NO: 1.

12. (canceled)

13. The recombinant genetic construct of claim 9, wherein the recombinant genetic construct further comprises:
a glial fibrillary acidic protein (GFAP) promoter-inclusive regulatory element, wherein said GFAP promoter-inclusive regulatory element is positioned within the recombinant genetic construct between the GPR17 promoter-inclusive regulatory element sequence and the nucleic acid sequence encoding the molecule of interest.

14. The recombinant genetic construct of claim 13, wherein the GFAP promoter-inclusive regulatory element has the sequence of SEQ ID NO: 3.

15. The recombinant genetic construct of claim 9, wherein the molecule of interest is a therapeutic molecule, or
is a post-transcriptional modulator of gene expression, said post-transcriptional modulator of gene expression being selected from the group consisting of an antisense oligonucleotide (ASO), small interfering RNA (siRNA), short or small hairpin RNA (shRNA), and microRNA (miRNA), or
is a phenoconversion-promoting molecule, said phenoconversion-promoting molecule being a neuronal reprogramming factor, or
is a gene-editing molecule, said gene-editing molecule being selected from the group consisting of an RNA-guided nuclease, a zinc finger nuclease, and a transcription activator-like effector nuclease (TALEN), or
is an epigenetic editing molecule, said epigenetic editing molecule being selected from the group consisting of a DNA methyltransferase enzyme, a histone demethylation enzyme, a histone methyltransferase enzyme, a transcription factor recruitment domain, and a zinc finger transcriptional repressor domain.

16-19. (canceled)

20. The recombinant genetic construct of claim 9, wherein the nucleic acid sequence encodes (i) a first molecule of interest and (ii) a second nucleic acid sequence encoding a second molecule of interest.

21. The recombinant genetic construct of claim 20 wherein the first molecule of interest and the second molecule of interest are each polypeptides.

22. The recombinant genetic construct of claim 21, wherein the recombinant nucleic acid construct further comprises:
a self-cleaving peptide encoding nucleotide sequence, wherein said self-cleaving peptide encoding sequence is positioned between the first nucleic acid sequence and the second nucleic acid sequence.

23-25. (canceled)

26. An expression vector comprising the recombinant genetic construct of claim 9.

27-28. (canceled)

29. A pharmaceutical composition comprising:
the recombinant genetic construct of claim 9 and
a pharmaceutically acceptable carrier.

30. A preparation of cells comprising glial progenitor cells, wherein the cells of the preparation comprise the recombinant genetic construct of claim 9.

31-33. (canceled)

34. A method of delivering a nucleic acid construct encoding a protein of interest to glial progenitor cells, said method comprising:
providing a recombinant genetic construct of claim 9
wherein said nucleic acid sequence encodes the protein of interest and is heterologous to the GPR17 promoter-inclusive regulatory element and wherein said nucleic acid sequence encoding the protein of interest is positioned 3′ to the GPR17 promoter-inclusive regulatory element sequence and
transfecting or transducing the glial progenitor cells with the recombinant genetic construct.

35. The method of claim 34, wherein the GPR17 promoter-inclusive regulatory element comprises the sequence of SEQ ID NO: 2 or SEQ ID NO: 1.

36-40. (canceled)

41. A method of delivering a nucleic acid construct encoding a protein of interest to astrocytes and glial progenitor cells, said method comprising:
providing a recombinant genetic construct comprising:
(i) a G-protein coupled receptor (GPR17) promoter-inclusive regulatory element sequence having a 5′ and a 3′ end;
(ii) a glial fibrillary acidic protein (GFAP) promoter-inclusive regulatory element; and
(ii) a nucleic acid sequence encoding the protein of interest, wherein said nucleic acid sequence encoding the protein of interest is heterologous to the GPR17 promoter-inclusive regulatory element as well as to the GFAP promoter-inclusive regulatory element and wherein said GFAP promoter-inclusive regulatory element is positioned within the recombinant genetic construct between the GPR17 promoter-inclusive regulatory element sequence and the nucleic acid sequence encoding the protein of interest and
transfecting or transducing a population of glial progenitor cells with the recombinant genetic construct, wherein
(a) prior to differentiation of the transfected or transduced glial progenitor cells, the nucleic acid sequence encoding said protein of interest is expressed under control of the GPR17 promoter-inclusive regulatory element and
(b) after differentiation of the transfected or transduced glial progenitor cells to astrocytes, said protein of interest is expressed under control of the GFAP promoter-inclusive regulatory element.

42. The method of claim 41, wherein the GPR17 promoter-inclusive regulatory element comprises the sequence of SEQ ID NO: 2 or SEQ ID NO: 1.

43-48. (canceled)


SEQUENCE LISTING (11 sequences)

SEQ ID NO: 1 - DNA, 2199 bp, synthetic construct
  source 1..2199: mol_type = other DNA
  regulatory 1..2199: regulatory_class = promoter
  misc_feature 1..2199: note = GPR17 Promoter
ggatacggaagagatccaatcgcagacccaagatccccacccaggttatggtgggcagaccccagatgccagggccacccattcagcatccctccctggaccccaggacctgctactgctgggtgtctggactccatcctgcacagcactgtgctccatctgccctggggtgtctcatcatcagctgtgtgcagggcaaggggcccaaacaaaggcccagcagtcactggctaagctgccgactggctctctgtgcctccccaagaccctatgtgcccagcagggggcaacagctcagggtcagctgaccgaatgcctcggtgaatgaatgactctacaagagaggaagggagcctcggtgggcatcatctcccctcgactactggccagagccctggctcttacaccccagcgacgggaagcagttgtggcctgtggcttcagtcttcatcaccacaatccctgaagcccacccttgcccagacacctgtgccccagccccaaccccaggccacctcctcagcaggtctggggctgagctgccccacctggcgcctatggcggccagcccatgccccctgcggtgcctctgtcccagactcagcatgtaggccccatgaccccactccacattctggtgactcctcctgagcgtcaggacaacactcaacccacgaggaattatttctgtctcaaagatgcaggaatcagctcaacgcctcaaaactccatcaccacggtcaatgcccttgaagccatcgacagtgatcaccccaataacagaaggtctgtgagcccagaaatgccctgctcagggtggttagcttcaagccaccacctttccaaccagcctgggccagttcttccagacagccgcctgcgggcacaacaggaaagagacctgcgccccggctcagacacctcacacccagctggctctcaggccagacaaactgggaagcccatctctcttgaaggaagtccagatgggaaacagcttctcaacagaccagatcacagcatcagatctaaaggtggccttcagaattctttttcaggttgaattaggatcaaatctaagaattctaaattcaaaatgcagcagaaaaacaaaacacacacacacacggagcctaagttctggagtgacatgtgcttgggttcaaatcctggctctgttgcttcctactgtttgttgatgggtgagtttcttcatttgcctgagcctcagtttccttgtctgtaaaatggggcaataatcccagctgcacagggtgatgtgaagagacaaatttaagacactgccccttaaatgctagccacatacatacagttttcaatgtttaaacaacaaaatgtaaagtcttttgaaaccaggaagggtgatttggtttcccatgttgctggatgtatcattttcagaaagacagagagaaatgaactttgttcactcagtctcagaggcggccgccggcagcattcaaaggcaccccagcccggagccaccccagggaggagccccaggccagcggtcagattcatgggcttccgtgcagaaggggagctgcaccggcgagcacccggcctctgagctgagccgcatcctcacggacaggacagcgccccattatgaggctcctgcagctgttcctcgctccagataaaggccatgatttattctgtgtgcccaaatggggcctcattatacagggcaggacacaaggaccctacagcaagtgtcctcaaagagtcgcctctcactccgtgagcaagactcctcggcctcccaccctccgttcacaggccccctccgccgtctgcgggcgcaggcctgggagcgccgcctgttgccatgacagccggcccctccctgccccccatcagtaggaaatcatccccttctgaaacgtcctgttgtgtccctcagctccagcccaagccccccacccagcccccgcctgctctgagtctctgagacagtcacacactcagactatgtggccaagctgggggcggggggcatgggctagggacacactagaatattcacgctccggtggcagcagcagcagcagccagaggagcagcccgacacaacaagggacccctcaggaatgaagcagcctttcagggccagaggggctgtggtctcccttcctctccttaaatagccagcgttccacccacagcggcaagggc

SEQ ID NO: 2 - DNA, 799 bp, synthetic construct
  source 1..799: mol_type = other DNA
  regulatory 1..799: regulatory_class = promoter
  misc_feature 1..799: note = GPR17 Promoter
gttgctggatgtatcattttcagaaagacagagagaaatgaactttgttcactcagtctcagaggcggccgccggcagcattcaaaggcaccccagcccggagccaccccagggaggagccccaggccagcggtcagattcatgggcttccgtgcagaaggggagctgcaccggcgagcacccggcctctgagctgagccgcatcctcacggacaggacagcgccccattatgaggctcctgcagctgttcctcgctccagataaaggccatgatttattctgtgtgcccaaatggggcctcattatacagggcaggacacaaggaccctacagcaagtgtcctcaaagagtcgcctctcactccgtgagcaagactcctcggcctcccaccctccgttcacaggccccctccgccgtctgcgggcgcaggcctgggagcgccgcctgttgccatgacagccggcccctccctgccccccatcagtaggaaatcatccccttctgaaacgtcctgttgtgtccctcagctccagcccaagccccccacccagcccccgcctgctctgagtctctgagacagtcacacactcagactatgtggccaagctgggggcggggggcatgggctagggacacactagaatattcacgctccggtggcagcagcagcagcagccagaggagcagcccgacacaacaagggacccctcaggaatgaagcagcctttcagggccagaggggctgtggtctcccttcctctccttaaatagccagcgttccacccacagcggcaaggg

SEQ ID NO: 3 - DNA, 682 bp, synthetic construct
  source 1..682: mol_type = other DNA
  regulatory 1..682: regulatory_class = promoter
  misc_feature 1..682: note = GFAP Promoter
acatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcgg

SEQ ID NO: 4 - DNA, 3122 bp, synthetic construct
  source 1..3122: mol_type = other DNA
  regulatory 1..2199: regulatory_class = promoter
  regulatory 2251..2932: regulatory_class = promoter
  misc_feature 1..3122: note = Dual GPR17/GFAP Promoter
  intron 2205..2247
  intron 2933..3122
ggatacggaagagatccaatcgcagacccaagatccccacccaggttatggtgggcagaccccagatgccagggccacccattcagcatccctccctggaccccaggacctgctactgctgggtgtctggactccatcctgcacagcactgtgctccatctgccctggggtgtctcatcatcagctgtgtgcagggcaaggggcccaaacaaaggcccagcagtcactggctaagctgccgactggctctctgtgcctccccaagaccctatgtgcccagcagggggcaacagctcagggtcagctgaccgaatgcctcggtgaatgaatgactctacaagagaggaagggagcctcggtgggcatcatctcccctcgactactggccagagccctggctcttacaccccagcgacgggaagcagttgtggcctgtggcttcagtcttcatcaccacaatccctgaagcccacccttgcccagacacctgtgccccagccccaaccccaggccacctcctcagcaggtctggggctgagctgccccacctggcgcctatggcggccagcccatgccccctgcggtgcctctgtcccagactcagcatgtaggccccatgaccccactccacattctggtgactcctcctgagcgtcaggacaacactcaacccacgaggaattatttctgtctcaaagatgcaggaatcagctcaacgcctcaaaactccatcaccacggtcaatgcccttgaagccatcgacagtgatcaccccaataacagaaggtctgtgagcccagaaatgccctgctcagggtggttagcttcaagccaccacctttccaaccagcctgggccagttcttccagacagccgcctgcgggcacaacaggaaagagacctgcgccccggctcagacacctcacacccagctggctctcaggccagacaaactgggaagcccatctctcttgaaggaagtccagatgggaaacagcttctcaacagaccagatcacagcatcagatctaaaggtggccttcagaattctttttcaggttgaattaggatcaaatctaagaattctaaattcaaaatgcagcagaaaaacaaaacacacacacacacggagcctaagttctggagtgacatgtgcttgggttcaaatcctggctctgttgcttcctactgtttgttgatgggtgagtttcttcatttgcctgagcctcagtttccttgtctgtaaaatggggcaataatcccagctgcacagggtgatgtgaagagacaaatttaagacactgccccttaaatgctagccacatacatacagttttcaatgtttaaacaacaaaatgtaaagtcttttgaaaccaggaagggtgatttggtttcccatgttgctggatgtatcattttcagaaagacagagagaaatgaactttgttcactcagtctcagaggcggccgccggcagcattcaaaggcaccccagcccggagccaccccagggaggagccccaggccagcggtcagattcatgggcttccgtgcagaaggggagctgcaccggcgagcacccggcctctgagctgagccgcatcctcacggacaggacagcgccccattatgaggctcctgcagctgttcctcgctccagataaaggccatgatttattctgtgtgcccaaatggggcctcattatacagggcaggacacaaggaccctacagcaagtgtcctcaaagagtcgcctctcactccgtgagcaagactcctcggcctcccaccctccgttcacaggccccctccgccgtctgcgggcgcaggcctgggagcgccgcctgttgccatgacagccggcccctccctgccccccatcagtaggaaatcatccccttctgaaacgtcctgttgtgtccctcagctccagcccaagccccccacccagcccccgcctgctctgagtctctgagacagtcacacactcagactatgtggccaagctgggggcggggggcatgggctagggacacactagaatattcacgctccggtggcagcagcagcagcagccagaggagcagcccgacacaacaagggacccctcaggaatgaagcagcctttcagggccagaggggctgtggtctcccttcctctccttaaatagccagcgttccacccacagcggcaagggcgcgccggagtcgctgcgcgctgccttcgccccgtgccccgctccgccgtcgacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggtcgacccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 5 - DNA, 66 bp, synthetic construct
  source 1..66: mol_type = other DNA
  misc_feature 1..66: note = Porcine teschovirus-1 2A (P2A)
ggaagcggagctactaacttcagcctgctgaagcaggctggagacgtggaggagaaccctggacct

SEQ ID NO: 6 - DNA, 66 bp, synthetic construct
  source 1..66: mol_type = other DNA
  misc_feature 1..66: note = Porcine teschovirus-1 2A (P2A), codon optimized
ggttccggagccacgaacttctctctgttaaagcaagcaggagacgtggaagaaaaccccggtccc

SEQ ID NO: 7 - DNA, 75 bp, synthetic construct
  source 1..75: mol_type = other DNA
  misc_feature 1..75: note = Foot and mouth disease virus 2A (F2A)
ggaagcggagtgaaacagactttgaattttgaccttctcaagttggcgggagacgtggagtccaaccctggacct

SEQ ID NO: 8 - DNA, 54 bp, synthetic construct
  source 1..54: mol_type = other DNA
  misc_feature 1..54: note = Thosea asigna virus 2A (T2A)
gagggcagaggaagtcttctaacatgcggtgacgtggaggagaatcccggccct

SEQ ID NO: 9 - DNA, 69 bp, synthetic construct
  source 1..69: mol_type = other DNA
  misc_feature 1..69: note = Equine rhinitis A virus 2A (E2A)
ggaagcggacagtgtactaattatgctctcttgaaattggctggagatgttgagagcaaccctggacct

SEQ ID NO: 10 - DNA, 66 bp, synthetic construct
  source 1..66: mol_type = other DNA
  misc_feature 1..66: note = Cytoplasmic polyhedrosis virus (BmCPV 2A)
gacgtttttcgctctaattatgacctactaaagttgtgcggtgatatcgagtctaatcctggacct

SEQ ID NO: 11 - DNA, 66 bp, synthetic construct
  source 1..66: mol_type = other DNA
  misc_feature 1..66: note = Flacherie virus (BmIFV 2A)
actctgacgagggcgaagattgaggatgaattgattcgtgcaggaattgaatcaaatcctggacct